(19) United States
(12) Patent Application Publication
(10) Pub. No.: US 2022/0125838 A1
(43) Pub. Date: Apr. 28, 2022

(54) IMMUNOMODULATORY GLYCOSPHINGOLIPIDS AND METHODS OF USE THEREOF

(71) Applicants: President and Fellows of Harvard College, Cambridge, MA (US); Seoul National University R&DB Foundation, Seoul (KR)

(72) Inventors: Sungwhan Oh, Cambridge, MA (US); Dennis L. Kasper, Cambridge, MA (US); Heebum Song, Seoul (KR); Seung Bum Park, Arlington, MA (US)

(73) Assignees: President and Fellows of Harvard College, Cambridge, MA (US); Seoul National University R&DB Foundation, Seoul (KR)

(21) Appl. No.: 17/427,756

(22) PCT Filed: Jan. 15, 2020
(86) PCT No.: PCT/US2020/013741

§ 371 Date: Aug. 2, 2021

Related U.S. Application Data
(60) Provisional application No. 62/803,941, filed on Feb. 11, 2019.

(51) Int. Cl. A61K 35/17; C07H 15/04; A61K 47/54; A61K 47/64; A61K 45/06; A61P 37/06

(52) U.S. Cl. CPC A61K 35/17; C07H 15/04; A61P 37/06; A61K 47/6425; A61K 45/06; A61K 47/549

(57) ABSTRACT

Provided herein are a subset of alpha-galactosylceramide (alpha-GC) compounds having improved immunomodulatory activity, particularly with respect to NKT cell number and activity. Also provided herein are methods of use of such compounds, including in the modulation of NKT cells and/or activity in vivo. Further provided are combinatorial synthesis methods for generating alpha-GC compounds of specifically defined structure and thereby generating pure preparations thereof.

### FEDERALLY SPONSORED RESEARCH

This invention was made with government support under grant numbers DK102771 and AT010268 awarded by the National Institutes of Health. The government has certain rights in this invention.

### BACKGROUND OF INVENTION

Invariant natural killer T (NKT) cells are important in both innate and the adaptive immunity. Once activated, they release massive amounts of inflammatory cytokines and play an important pro-inflammatory role in a range of disease processes, including autoimmune diseases, infectious diseases, and cancer. Commensal microbiota can modulate NKT cell numbers in vivo, including in the gut and in the lungs. As a result, conventionally colonized mice (e.g., the normal mouse microbiome) have significantly fewer NKT cells in these two compartments compared to germ-free mice. When challenged in NKT cell-dependent asthma and ulcerative colitis models, the conventional mice are protected while the germ-free mice had severe disease phenotypes, suggesting that low numbers of NKT cells in the colon and lung are associated with resistance to experimental colitis and asthma.

### SUMMARY OF INVENTION

The invention is premised on the unexpected finding that certain structurally defined glycosphingolipids have improved activity. One particularly unexpected finding of the invention is the discovery of a specific subclass and specific species of glycosphingolipids, defined by a particular structure, having greater ability to inhibit invariant natural killer T (NKT) cells as compared to a broader class of glycosphingolipids. This immune inhibitory class of glycosphingolipids are characterized as a subset of alpha-galactosylceramides (alpha-GC) having branched sphingoid chains, and particularly those having branched sphinganine chains. It has been found that these compounds are better able to inhibit NKT activation and reduce NKT cell numbers as compared to alpha-GC having linear sphinganine chains. It was not previously known that alpha-GC having branched sphinganine chains were more potent than alpha-GC having linear sphinganine chains. Previous work reported similar activity levels in alpha-GC having branched or unbranched sphinganine or fatty acid chains. The invention therefore contemplates the use of these more potent compounds in treating or preventing conditions characterized by increased NKT cell numbers and/or activity including without limitation autoimmune disorders such as colitis.

Thus, in one embodiment, the invention provides an immune inhibitory alpha-galactosylceramide (referred to herein as alpha-GC for brevity) having a branched sphinganine chain. The alpha-GC comprises a galactose head group and a ceramide which in turn comprises a fatty acid chain and a sphinganine chain that is branched, preferably terminally branched.

In some embodiments, the alpha-GC comprises a sphinganine chain having a length of 17-19 carbons. The sphinganine chain, in some embodiments, is 17 carbons in length. The fatty acid (acyl) chain length may range from 15-17 carbons. In some embodiments, the sphinganine chain length is 17 carbons and the fatty acid chain length is 15, 16 or 17 carbons.

In some embodiments, the alpha-GC comprises a fatty acid chain having one or more hydroxyl groups. In some embodiments, the fatty acid chain has one hydroxyl group. In some embodiments, the fatty acid chain has a hydroxyl group on C2 (sometimes referred to as C2′ to denote position on the fatty acid chain rather than the sphinganine chain). In some embodiments, the fatty acid chain has a hydroxyl group on C3 (sometimes referred to as C3′ to denote position on the fatty acid chain rather than the sphinganine chain). In some embodiments, the sphinganine chain has a hydroxyl group at the C4 position (sometimes referred to as C4′ to denote position on the fatty acid chain rather than the sphinganine chain). In some embodiments, the isolated alpha-GC comprises a sphinganine chain having one or more hydroxyl groups. Hydroxyls on the sphinganine chain can occur at positions C2, C3 or C4, in some embodiments.

Significantly, the alpha-GC comprises a sphinganine chain that is branched. Branching preferably is terminal branching. The sphinganine chain may be branched at the omega-2 (iso) position or omega-3 (ante-iso) position. Thus, the sphinganine chain may comprise a terminal isomethyl or an ante-isomethyl group.

The fatty acid chain similarly be branched, although it is not so required for the more potent activity observed and reported herein. Thus, in some embodiments, each of the fatty acid or sphinganine chains comprises a terminal isomethyl or an ante-isomethyl group.

In some embodiments, the alpha-GC has a linear fatty acid chain and a sphinganine chain having a terminal isomethyl group. In some embodiments, the alpha-GC has a linear fatty acid chain and a sphinganine chain having a terminal ante-isomethyl group.

The alpha-GC may be provided in an isolated or pure form. When provided in compositions, the alpha-GC may be provided in varying degrees of purity, depending on the degree of their separation from other components including for example components from an in vitro synthesis environment such as that disclosed herein. The alpha-GC may represent for example at least 50%, at least 60%, at least 70%, at least 80%, at least 90%, at least 95%, at least 99% (w/w) of the composition, or in other embodiments, it may represent 100% of the composition (w/w) indicating complete purity. In some embodiments, the alpha-GC provided herein is produced synthetically and is not structurally identical to any naturally occurring alpha-GC. One or more of the alpha-GC compounds provided herein may or may not be naturally occurring. In some embodiments, the compositions provided herein comprise non-naturally occurring alpha-GC compounds. The methods of use provided herein may or may not use a naturally occurring alpha-GC compound. In some embodiments, such alpha-GC compounds may be isolated or purified to a degree as provided herein.

In some embodiments, a carbon of either or both the fatty acid or the sphinganine that is attached to a hydroxyl group exhibits chirality (and is therefore referred to as a chiral carbon). Such chirality may be R-chirality or S-chirality.

In some embodiments, the fatty acid C2 position has R-chirality. In some embodiments, the fatty acid C2 position has S-chirality. In some embodiments, the isolated alpha-GC is present as a racemic mixture at the fatty acid C2 position. In some embodiments, the fatty acid C3 position has R-chirality. In some embodiments, the fatty acid C3 position has S-chirality. In some embodiments, the isolated alpha-GC is present as a racemic mixture at the fatty acid C3 position. In some embodiments, the fatty acid C4 position has R-chirality. In some embodiments, the fatty acid C4 position has S-chirality. In some embodiments, the isolated alpha-GC is present as a racemic mixture at the fatty acid C4 position.

In some embodiments, the sphinganine C2 position has R-chirality. In some embodiments, the sphinganine C2 position has S-chirality. In some embodiments, the isolated alpha-GC is present as a racemic mixture at the sphinganine C2 position. In some embodiments, the sphinganine C3 position has R-chirality. In some embodiments, the sphinganine C3 position has S-chirality. In some embodiments, the isolated alpha-GC is present as a racemic mixture at the sphinganine C3 position. In some embodiments, the sphinganine C4 position has R-chirality.

In some embodiments, the sphinganine C4 position has S-chirality. In some embodiments, the isolated alpha-GC is present as a racemic mixture at the sphinganine C4 position.

In another aspect, the invention provides a compound of formula (I):

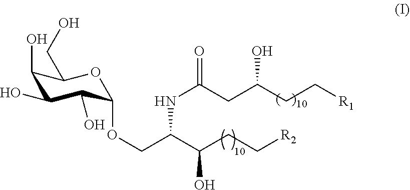

or an enantiomer or diastereomer thereof, wherein, R₁ is methyl, ethyl, or branched or unbranched C₃-C₆ alkyl and R₂ is are methyl or branched C₃-C₆ alkyl.

In another aspect, the invention provides a compound of formula (I):

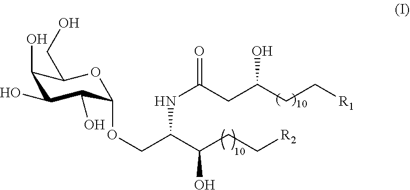

or an enantiomer or diastereomer thereof, wherein, R₁ is methyl, ethyl, or branched or unbranched C₁-C₅ alkyl and R₂ is are methyl or branched C₂-C₅ alkyl. In certain embodiments, the compound of formula (I) is an isolated compound. In certain embodiments, the compound is at least 95% pure.

In certain embodiments, the compound of formula (I) is an isolated compound. In certain embodiments, the compound is at least 95% pure.

In certain embodiments, the compound of formula (I) is selected from the compounds of FIG. 1G.

Another aspect of this disclosure provides an isolated compound having the structure of formula (I):

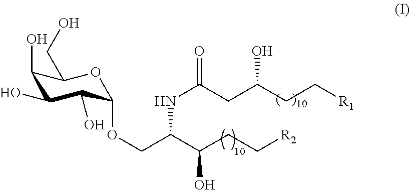

or an enantiomer or diastereomer thereof, wherein:

(a) R₁ and R₂ independently are methyl, ethyl, or branched or unbranched C₃-C₆ alkyl, or

(b) R₁ is methyl, ethyl, or branched or unbranched C₃-C₆ alkyl and R₂ is methyl or branched C₃-C₆ alkyl, or

(c) R₁ is methyl, ethyl, or branched or unbranched C₁-C₅ alkyl and R₂ is methyl, ethyl, or branched or unbranched C₂-C₅ alkyl, or

(d) R₁ is methyl, ethyl, or branched or unbranched C₁-C₅ alkyl and R₂ is methyl or branched C₂-C₅ alkyl.

In some embodiments, R₂ is a branched alkyl.

In some embodiments, R₁ is selected from the group consisting of methyl, ethyl, n-propyl, isopropyl, n-butyl, sec-butyl, and n-pentyl.

In some embodiments, R₂ is independently selected from the group consisting of methyl, n-propyl, isopropyl, n-butyl, sec-butyl, and n-pentyl.

In some embodiments, R₁ is selected from methyl, ethyl, isopropyl, and sec-butyl; and R₂ is selected from isopropyl, n-butyl, sec-butyl, and n-pentyl.

In some embodiments, both of R₁ and R₂ are branched, or R₁ is unbranched and R₂ is branched.

In some embodiments, the compound is substantially free of homologues, positional isomers, and stereoisomers.

In some embodiments, the compound is at least 95% pure.

In some embodiments, R₂ is methyl or branched C₃-C₆ alkyl. In some embodiments, R₂ is methyl or branched C₂-C₅ alkyl.

Specific compounds identified as having improved and unexpected potency are as follows:

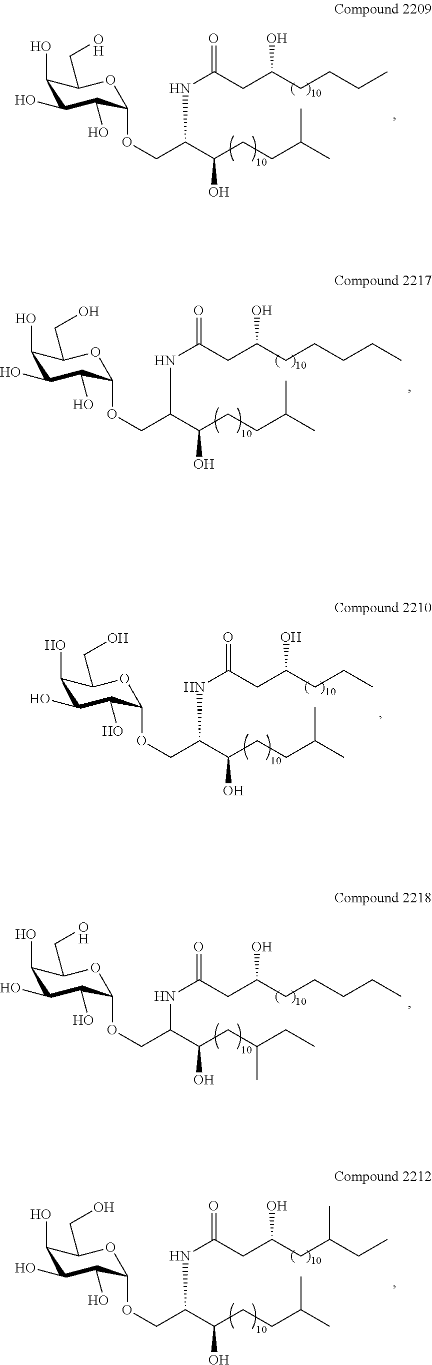

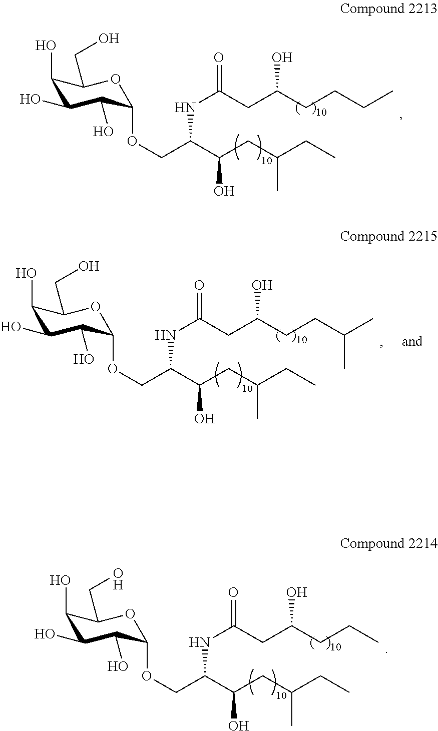

In some embodiments, the compounds may be:

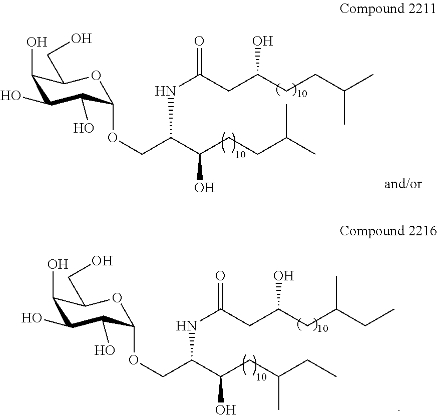

In other embodiments, the compounds (also referred to herein as alpha-GC compounds) exclude compounds having terminally branched sphinganine comprising an isomethyl and terminally branched acyl chain comprising an isomethyl (e.g., as such terminal groups are shown in Compound 2211) or compounds having terminally branched sphinganine comprising an ante-isomethyl and terminally branched acyl chain comprising an ante-isomethyl (e.g., as such terminal groups are shown in Compound 2216).

In some embodiments, any of the foregoing compounds are used individually. In other embodiments, combinations (i.e., mixtures) of these compounds may be used. For example, 2, 3, 4, 5 or more of the compounds may be provided as a mixture.

In some embodiments, the alpha-GC is complexed or conjugated to another moiety. One such moiety may be a CD1d protein. The alpha-GC may be non-covalently complexed to CD1d protein. Compositions comprising alpha-GC do not comprise bacterial membrane or bacterial membrane components, and also do not contain other bacterial components such as LPS and/or other glycolipids.

In some embodiments, the alpha-GC is provided in a lyophilized form. Lyophilized forms are particularly suitable for long-term storage, ranging from days, weeks, months or even years. In some embodiments, the glycolipid is provided in a form that is suitable for administration to a human, including an orally administered form or a parenterally administered form.

This disclosure further provides compositions comprising any of the foregoing alpha-GC. In some embodiments, the composition is sterile. In some embodiments, the composition is intended for in vivo use in human or animal subjects. In some embodiments, the composition is intended for in vitro use. The composition may comprise preservatives, including preferably a non-naturally occurring preservative. The composition may comprise thimerosal, aluminum hydroxide, benzethonium chloride, formaldehyde, formalin, glutaraldehyde, potassium phosphate, aluminum potassium sulfate, bovine extract, calf serum, ammonium sulfate, aluminum phosphate, non-human cells, Vero (monkey kidney) cells, human cells, MRC-5 (human diploid) cells, MRC-5 cellular proteins, and the like. The composition may comprise a non-naturally occurring stabilizer. The composition may comprise human albumin, phenol, glycerin or glycine. The alpha-GC may be provided in an isolated form, including a pure form in some instances.

In another aspect, the invention provides a pharmaceutical composition comprising any of the foregoing alpha-GC. The pharmaceutical composition may further comprise a pharmaceutically acceptable carrier or it may be combined with a pharmaceutically acceptable carrier (e.g., it may be a lyophilized form of the alpha-GC).

In some embodiments, the isolated alpha-GC is formulated for delivery to lungs (e.g., with an atomizer or nebulizer). In some embodiments, the isolated alpha-GC is formulated for delivery to the gut or colon. In these latter instances, it may be formulated as a capsule, pill, tablet, gel, lozenge, oral solution, suspension, syrup, emulsion, elixir, or other ingestible form. Such solid forms may be controlled release forms including delayed release forms and/or extended release forms, including forms that release active agent in response to pH. Such solid forms may comprise an enteric coating. It may further be formulated as a suppository or an enema for rectal delivery.

In some embodiments, the composition or pharmaceutical composition further comprises another agent such as but not limited to an immunomodulatory agent such as an immunosuppressant or an anti-inflammatory agent or an agent that modulates Th1 and/or Th2 immune responses. The immunomodulatory agent may be one used to treat or prevent an inflammatory condition such as an autoimmune disease (e.g., colitis).

It is to be understood that the isolated alpha-GC may be provided in a pure form or as a mixture of different alpha-GC. The invention contemplates that the composition may be stereochemically pure or it may be a racemic mixture at one or more positions in the alpha-GC. Unless stated otherwise, the alpha-GC are non-naturally occurring alpha-GC.

In another aspect, the invention provides a method comprising administering to a subject having or at risk of developing a condition characterized by increased NKT cell numbers or activity any of the foregoing alpha-GC in an effective amount to decrease NKT cell numbers or activity and/or to prevent or treat the condition. The alpha-GC may be administered in an isolated form and/or a purified form.

In some embodiments, the condition is an inflammatory condition. In some embodiments, the condition is an autoimmune disease. In some embodiments, the condition is inflammatory bowel disease, such as but not limited to human juvenile inflammatory bowel disease (IBD). In some embodiments, the condition is colitis (e.g., ulcerative colitis). In some embodiments, the condition is systemic lupus erythematosus (i.e., lupus). In some embodiments, the condition is multiple sclerosis. In some embodiments, the condition is arthritis. In some embodiments, the condition is asthma. In some embodiments, the condition is arthritis. In some embodiments, the condition is inflammatory dermatitis.

In some embodiments, the alpha-GC is administered locally such as to the colon or gut or to the lungs using, for example, any of the formulations provided herein and/or known in the art. Local administration to the lungs may be carried out via nebulization, as an example. In some embodiments, the alpha-GC is administered systemically. In some embodiments, the alpha-GC is administered intraperitoneally.

In some embodiments, the subject is human. In some embodiments, the subject is less than 5 years of age, less than 1 year of age, less than 6 months of age, or less than 1 month of age. In some embodiments, the subject was delivered by cesarean section and/or was not breast-fed as an infant. In some embodiments, the subject is a pregnant subject and optionally the subject and/or the fetus is at high risk of developing a condition characterized by increased NKT cell numbers or activity. In some embodiments, the subject is a female subject of child-bearing age (e.g., in humans, approximately 15-55 years of age), and optionally is at increased (i.e., above-normal) risk of developing a condition characterized by increased NKT cell numbers or activity.

In some embodiments, the subject is administered a second active agent such as an immunosuppressant or an anti-inflammatory agent.

In some embodiments, the method further comprises identifying a subject having or at risk (including increased risk) of developing the condition. Such subjects may or may not have been diagnosed with a condition characterized by increased NKT cell number or activity.

In another aspect, the invention provides a method comprising contacting CD1d-expressing antigen presenting cells or NKT activating antigen presenting cells with any of the foregoing alpha-GC, and contacting the antigen presenting cells with activated NKT cells.

In some embodiments, the antigen presenting cells are dendritic cells.

In some embodiments, the antigen presenting cells are contacted with the alpha-GC in vitro. In some embodiments, the antigen presenting cells, loaded with alpha-GC, are contacted with the activated NKT cells in vivo.

In another aspect, the invention provides a method comprising contacting isolated CD1d protein loaded with (i.e., bound to) any of the foregoing alpha-GC with activated NKT cells. Isolated CD1d protein refers to CD1d that is not provided in the context of a cell such as an antigen-presenting cell. Contacting may occur in vitro or in vivo.

In another aspect, the invention provides a method comprising administering to a subject having or at risk of developing a condition characterized by increased NKT cell numbers or activity any of the foregoing alpha-GC bound to CD1d protein in an effective amount to decrease NKT cell number or activity, wherein the CD1d protein is isolated (i.e., not provided in a cell-bound form). The CD1d protein may be provided as a CD1d tetramer.

In another aspect, the invention provides method of making a compound of formula (I), comprising:

(1) condensing a compound of formula (II):

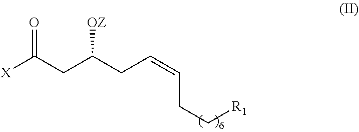

wherein X is —OH or a leaving group; and Z is an oxygen protecting group;
with a compound of formula (III):

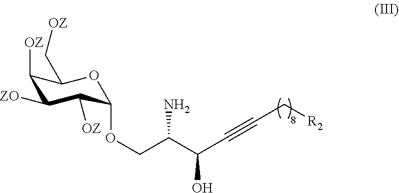

wherein each Z independently is an oxygen protecting group;

to afford a compound of formula (IV):

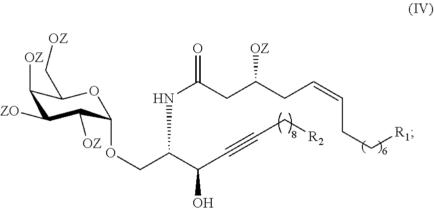

and

(2) reducing the unsaturated carbon-carbon bonds and deprotecting the protected hydroxyl groups to afford the compound of formula (I).

In certain embodiments, the method makes a plurality of compounds of formula (I), and comprises:

(1) condensing a plurality of compounds of formula (II) with a plurality of compounds of formula (III), to afford a plurality of compounds of formula (IV); and

(2) reducing the unsaturated carbon-carbon bonds and deprotecting the protected hydroxyl groups of the plurality of compounds of formula (IV) to afford the plurality of compounds of formula (I).

This disclosure further provides compounds produced according to the preceding methods. Such compounds may be provided in purity of at least 95% (w/w). Such compounds may be provided in combinations of 2, 3, 4, 5, 6, 7, 8, 9, 10, or more including 20, 30, 40, 50, 60, 70, 80, 90, 100 or more. The purity of such combinations may also be at least 95% (w/w) intending that at least 95% (w/w) of the composition is comprised of the structurally defined compounds, and does not include other compounds such as but not limited to bacterial cell membrane components, LPS, etc.

Other advantages and novel features of the present invention will become apparent from the following detailed description of various non-limiting embodiments of the invention when considered in conjunction with the accompanying Figures. In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

### BRIEF DESCRIPTION OF DRAWINGS

FIGS. 1A to 1K show structural assignments of BfaGCs. FIG. 1A: Extracted ion chromatogram (XIC) of prominent C34 BfaGCs ([M+CO₂⁻]—=762.57) showed that BfaGCs are isobaric mixtures separated by reverse-phase mobility. FIGS. 1B and 1C: MS²-XICs reveal co-eluting chemical homologues. Two isobaric species with aliphatic chains of C₁₇/C₁₇ (B) and C₁₈/C₁₆ (C) were assigned with MS/MS fingerprints of 490 and 504, respectively. FIG. 1D: MS/MS fingerprints of three peaks (retention times=11.0, 11.5, and 12.0, respectively) show a significant difference in relative intensity of MS/MS fragments of 490 (C₁₇/C₁₇) and 504 (C₁₈/C₁₆), implying that the 11.5- and 12.0-minute peaks are a mixture of chain-length homologues. FIG. 1E: Retrograde synthesis of BfaGC analogues. FIG. 1F: Co-injection analysis with synthetic BfaGCs determined that the number of terminal branches in acyl chains is positively correlated (less retention) with reverse-phase LC mobility. FIG. 1G: Structures of 16 synthetic BfaGCs (SB2201-SB2216) and the matrix-based synthesis scheme. FIG. 1H: Synthesis of the fatty acid building blocks. FIG. 1I: Synthesis of the alpha-galactosyl-sphingoid building blocks. FIG. 1J: Construction of the BfaGC library. FIG. 1K: Structures of 5 isobaric, C₁₇/C₁₇ BfaGCs.

FIGS. 2A to 2C show BCAA level dictates production of branched-chain BfaGCs. FIG. 2A: Ratios among differently branched C₃₄ BfaGCs are opposite between B. fragilis grown in rich medium and bacteria grown in minimal medium. Supplementation with individual BCAAs on defined medium increased production of branched-chain (both double and single) BfaGCs.

FIG. 2B: Host dietary BCAA is directly incorporated into BfaGCs in the large intestine. MS-XIC of d3- and d6-C₃₄BfaGC shows deuterium-labeled leucine is incorporated to BfaGC in the host gut. FIG. 2C: The MS/MS fingerprint (m/z=493) of d₆-C₃₄BfaGC confirms d3-leucine incorporation into both sphinganine and fatty acyl chains. FIG. 2D: Biosynthesis from BCAAs to branched-chain fatty acids (BCFAs). A set of bacterial enzymes—BCAA aminotransferase (BCAT), 2-oxoisovalerate dehydrogenase (BKDA/B), and elongase—are involved. Valine is converted to branched C4(isobutyl)-CoA, which is further converted to even-numbered isomethyl-BCFAs. Leucine and isoleucine are converted to branched C₅-CoAs (2- and 3-methylbutyrate, respectively), converted to odd-numbered isomethyl- or ante-isomethyl-BCFAs. FIG. 2E: MS/MS fragmentation confirmed that each BCAA is directly correlated with a specific chain length and specific branching in BfaGCs. Valine supplementation produces branched C₁₈/C₁₆ BfaGCs (fingerprint fragment m/z=504) as major products, whereas leucine and isoleucine supplementation produces C₁₇/C₁₇ BfaGCs (m/z=490, i17/i17 or a17/a17 respectively). *=unassigned background ion.

FIGS. 3A to 3D show branching of the sphinganine chain in BfaGCs directly impacts NKT cell recognition and activation. FIG. 3A: Panel screening of 21 synthetic ligands with BMDC-NKT cell co-culture (300 nM) found that sphinganine branching is a critical factor in NKT cell recognition (IL-2 production). K=KRN7000 (positive control), V=vehicle. FIG. 3B: In vivo IL-2 levels 2 h after aGC administration resemble those in in vitro profiles. FIGS. 3C and 3D: When injected intraperitoneally, sphinganine-branched SB2217 triggers neither Th1 (IFN-γ) nor Th2 (IL-4) cytokines in vivo, whereas unbranched SB2219 is a very weak Th1 agonist and a non-agonist of Th2 cytokines. All panels show mean±SEM values.

FIGS. 4A to 4E show sphinganine-branched BfaGC protects conventional mice from NKT cell-mediated colitis. FIG. 4A: Disease progression and monitoring scheme. Intraperitoneal injection of SB2217 protects mice, as judged by multiple criteria: survival curve (FIG. 4B), tissue histopathology (FIG. 4C), disease scores (FIG. 4D), and weight loss (mean±SEM) (FIG. 4E). FIGS. 4F and 4G show BCAA restriction/supplementation changes the ratio of branched-chain to straight-chain BfaGCs in the gut lumen. FIG. 4F: Animals ate a BCAA-deficient diet for 7 days and subsequently received 1% leucine-supplemented drinking water. FIG. 4G: Ratio of mono-/di-branched BfaGCs depends on BCAA availability in the diet. FIGS. 4H and 4I show administration of SB2217 protects germ-free mice from NKT cell-mediated colitis. FIG. 4H shows disease progression and monitoring scheme. SB2217 injected mice show less weight loss than in the vehicle-treated group. FIG. 4J shows host dietary BCAAs directly impact the structure of BfaGCs in the gut. These symbiotic metabolites regulate host NKT cell to protect the host from NKT cell-mediated inflammatory responses.

### DETAILED DESCRIPTION OF INVENTION

The invention provides compositions and methods of use of a newly defined class of immune inhibitory alpha-galactosylceramides (alpha-GC) having more robust immunomodulatory properties than heretofore recognized. As described in the Examples, in accordance with the invention, it was expectedly found that a subclass of alpha-GC are more potent inhibitors of NKT cells. This subclass is structurally defined as alpha-GC having a branched sphinganine chain, including a terminally branched sphinganine chain. These compounds were found to stimulate IL-2 release in vitro, in BMDC-NKT cell co-cultures, and in vivo. This finding indicated that sphinganine branching is an important factor for NKT cell recognition. Such activity resembles the activity of KRN7000 (a previously described alpha-GC having a C26 saturated chain) and OCH (a previously described alpha-GC having a C8 saturated chain). However, when administered intraperitoneally, these compounds do not stimulate the release of Th1 (e.g., IFN-gamma) or Th2 (e.g., IL-4) cytokines in vivo. This is in contrast to KRN70000 which stimulates IFN-gamma and OCH which stimulates IL-4. An alpha-GC representative of the less potent alpha-GC class, compound 2219, was found to be a very weak Th1 agonist and a non-agonist of Th2 cytokines.

The alpha-GC of this disclosure may be produced synthetically, as described in the Examples, or in some instances some may be produced using or derived from B. fragilis components.

An immunoinhibitory alpha-GC class was previously identified and broadly structurally defined to include branched and unbranched fatty acids and sphingosine chains. (As used herein, the terms unbranched, linear and straight-chain are used interchangeably.) Prior to the findings that form the basis of this disclosure, no particular significance was ascribed to any of the structural features of this previously described class.

Accordingly, it was surprisingly found that alpha-GC within this previously defined class had different activities and more surprisingly that the activities correlated with the presence or absence of a branched sphinganine chain. As described herein, those compounds having a branched sphinganine chain have more potent activity than do those compounds having an unbranched sphinganine chain. Of those compounds found to have more robust activity, all had terminal sphinganine chain branching.

The three most potent alpha-GC compounds had isomethyl groups on the free end of the sphinganine chain. These three compounds also had unbranched fatty acid chains, 15-17 carbons in length. Accordingly, one subclass of more potent alpha-GC compounds may be defined as having a sphinganine chain with an terminal isomethyl group and an unbranched fatty acid chain of 15-17 carbons in length. The sphinganine chain may be 17 carbons in length.

Still other compounds have a terminally branched sphinganine chain comprising an ante-isomethyl group.

In addition to their cytokine induction capacity in vitro and in vivo, these compounds have also been shown to be effective in animal models of colitis. As described in the Examples, compound 2217 was shown to protect conventional mice from NKT cell-mediated colitis as evidenced by reduced mortality, weight loss and colonic inflammation. This same compound was also shown to protect germ-free adult animals which having higher levels of colonic NKT cells and tend to develop more severe colitis. These findings indicate that the newly structurally defined class of more potent alpha-GC compounds is able to regulate NKT cells and protect hosts independent of the basal colonic NKT cell level and/or the colonization status of the host.

The invention therefore provides this newly defined class of immunoinhibitory alpha-GC compounds, compositions thereof, and in vitro and in vivo methods of use thereof.

Specific immunoinhibitory alpha-GC compounds provided by this disclosure include compounds 2209, 2217, 2210, 2218, 2212, 2213, 2216, 2215, 2214 and 2211, in order of their potency (from high to low). The structures of these compounds are provided herein.

When provided as compositions or preparations, these compounds may have varying degrees of purity, including for example 50% or more, 60% or more, 70% or more, 80% or more, 90% or more, 95% or more, 99% or more, or 100% purity. Purity may be determined by performing an chemical analysis on the preparation such as a mass spec or an HPLC in order to determine if other components are present in the preparation and to quantitate such components relative to the desired immunoinhibitory alpha-GC compound. Purity may be expressed in terms of [weight of the immunoinhibitory alpha-GC] over [weight of the preparation], preferably where the preparation is dried, lyophilized, and the like.

As used herein, an immunoinhibitory alpha-GC compound or preparation intends that the compound or preparation is able to inhibit or reduce NKT cell numbers and/or NKT cell activity. It is therefore to be understood that the immunoinhibitory compound and preparations thereof are immunoinhibitory in the context of activated NKT cells. For example, the compounds and preparations are able to reduce the number and activity of NKT cells, including activated NKT cells, and/or are able to prevent the activation of NKT cells in the presence of an agent that stimulates NKT cells such as an immunostimulatory alpha-GC such as for example KRN7000. Thus, in some instances, the immunoinhibitory alpha-GC of the invention are able to compete and/or interfere with immunostimulatory alpha-GC such as KRN7000, thereby preventing or reducing the degree of stimulation that would otherwise occur in the presence of KRN7000 alone. Where NKT cell numbers and activity levels are normal (e.g., the levels in a subject that does not have an inflammatory condition and/or is not at elevated risk of developing an inflammatory condition (as a result of heredity, for example)), then the immunoinhibitory compounds and preparations may manifest no immunoinhibitory effect essentially because there is no observable background NKT cell based immune stimulation. In some instances, however, they may manifest no immunoinhibitory effect in the short term but may function to prevent immunostimulation in the long term by rendering a subject or the offspring of a subject, such as an infant or a child, resistant to future aberrant NKT cell based stimulation.

Assays for measuring NKT cell numbers are known in the art. Assays for measuring NKT cell activity are also known in the art. See for example US Patent Application Publication No. US 2015/0252068. These assays include cytokine production assays such IL-2 production assays. In some instances, a reduction or inhibition of NKT cell numbers and/or NKT cell activity is measured by symptoms that result from increased numbers of NKT cells and/or increased NKT cell activity. Such symptoms include the those associated with inflammatory conditions such as but not limited to autoimmune diseases. An exemplary but not limiting inflammatory condition is asthma. An exemplary but not limiting autoimmune disease is colitis.

This newly defined class of alpha-GC comprise sphinganine chains that are branched. The fatty acid chains, on the other hand, may be branched or unbranched. Branching may occur at one or more positions on the sphinganine chains and optionally the fatty acid chain. The branch may be of any length. In some instances, the branch point is at the penultimate carbon in the chain (referred to as the omega-2 or iso position). In some instances, the branch point is at the 3^{rd} last carbon in the chain (referred to as omega-3 or anteiso position).

The fatty acid and sphinganine chain may each independently comprise one or more hydroxyl groups. The position of the hydroxyl group may vary. In some instances, the hydroxyl group may be at the C2 position of the fatty acid chain. In some instances, the hydroxyl group may be at the C2 position of the sphinganine chain. In some instances, hydroxyl groups may be at both the C2 of the fatty acid and the C2 of the sphinganine. In some instances, the hydroxyl group may be at the C3 position of the fatty acid chain. In some instances, the hydroxyl group may be at the C3 position of the sphinganine chain. In some instances, hydroxyl groups may be at both the C3 of the fatty acid and the C3 of the sphinganine. In some instances, the hydroxyl group may be at the C4 position of the fatty acid chain. In some instances, the hydroxyl group may be at the C4 position of the sphinganine chain. In some instances, hydroxyl groups may be at both the C4 of the fatty acid and the C4 of the sphinganine. Any combination of hydroxyl substitutions at positions C1-C4 of the fatty acid and C1-C4 of the sphinganine chain are contemplated by the invention.

The alpha-GC may be provided as pure isomers or isomeric mixtures. Molecules that have the same molecular formula but differ in the nature or sequence of bonding of their atoms or the arrangement of their atoms in space are termed isomers. Isomers that differ in the arrangement of their atoms in space are termed stereoisomers. Stereoisomers that are not mirror images of one another are termed diastereomers and those that are non-superimposable mirror images of each other are termed enantiomers. When a molecule has an asymmetric center that, for example, is bonded to four different groups, then a pair of enantiomers is possible. An enantiomer can be characterized by the absolute configuration of its asymmetric center and can be described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing both enantiomers is called a “racemic mixture”.

In the case of the alpha-GC of this disclosure, asymmetric centers are possible at least at carbons bonded to hydroxyl groups. At each such asymmetric carbon, the chirality may be an R or an S configuration. Any combination of chiralities at the various chiral carbons in the molecule are contemplated by the invention.

As an example, the C3 position in the fatty acid, if bonded to a hydroxyl, may be in the R configuration or the S configuration or alternatively the molecule as a whole may exist as a racemic mixture of molecules having either R or S configuration at that position. Similarly, the C3 position in the sphinganine chain, if bonded to a hydroxyl, may be in the R configuration or the S configuration or alternatively the molecule as a whole may exist as a racemic mixture of molecules having either R or S configuration at that position. The invention therefore contemplates every combination of chirality at these positions including R—R, R—S, S—S and S—R (i.e., chirality of the fatty acid C3 position and chirality of the sphinganine C3 position).

As another example, the C2 position in the fatty acid, if bonded to a hydroxyl, may be in the R configuration or the S configuration or alternatively the molecule as a whole may exist as a racemic mixture of molecules having either R or S configuration at that position. The C2 position in the sphinganine chain, if bonded to a hydroxyl, may be in the R configuration or the S configuration or alternatively the molecule as a whole may exist as a racemic mixture of molecules having either R or S configuration at that position. The invention therefore contemplates every combination of chirality at these positions including R—R, R—S, S—S and S—R (i.e., chirality of the fatty acid C2 position and chirality of the sphinganine C2 position).

As another example, the C4 position in the fatty acid, if bonded to a hydroxyl, may be in the R configuration or the S configuration or alternatively the molecule as a whole may exist as a racemic mixture of molecules having either R or S configuration at that position. The C4 position in the sphinganine chain, if bonded to a hydroxyl, may be in the R configuration or the S configuration or alternatively the molecule as a whole may exist as a racemic mixture of molecules having either R or S configuration at that position. The invention therefore contemplates every combination of chirality at these positions including R—R, R—S, S—S and S—R (i.e., chirality of the fatty acid C4 position and chirality of the sphinganine C4 position).

The term polymorphs refers to a crystalline form of a compound (or a salt, hydrate, or solvate thereof) in a particular crystal packing arrangement. All polymorphs have the same elemental composition. Different crystalline forms usually have different X-ray diffraction patterns, infrared spectra, melting points, density, hardness, crystal shape, optical and electrical properties, stability, and solubility. Recrystallization solvent, rate of crystallization, storage temperature, and other factors may cause one crystal form to dominate. Various polymorphs of a molecule can be prepared by crystallization under different conditions. This disclosure contemplates various polymorphs of the compounds disclosed herein, including when complexed with another entity such as but not limited to CD1d.

The alpha-GC of the invention may be isolated. As used herein, the term isolated means partial or complete physical separation of the moiety of interest (e.g., the alpha-GC) from other moieties. As an example, if an alpha-GC can be obtained from a naturally occurring source, the isolated alpha-GC may be physically separated from the environment in which it naturally occurs (e.g., a B. fragilis cell or membrane). In the case of alpha-GC that are synthesized ex vivo, an isolated alpha-GC may be physically separated from the reaction mixture in which it was synthesized.

The alpha-GC compounds of this disclosure, whether used individually or as a mixture of structurally different compounds, can be used alone or together with CD1d molecules. CD1d proteins bind to these compounds and facilitate their presentation to NKT cells. Cells capable of presenting the alpha-GC compounds are therefore typically regarded as CD1d-positive antigen presenting cells. Examples of such cells include bone marrow derived dendritic cells (BMDC, as used in the Examples). Certain aspects of this disclosure therefore contemplate use, including administration, of immunoinhibitory alpha-GC when coupled (or bound or complexed) with isolated CD1d protein (i.e., CD1d protein that is not provided as an CD1d-bearing antigen presenting cell). In some instances, the CD1d protein is provided as a tetramer.

Without intending to be bound by any particular theory or underlying mechanism, the disclosure contemplates, inter alia, that the alpha-GC provided herein are able to compete with immunostimulatory alpha-GC for binding to CD1d and in this way reduce the efficacy of the immunostimulatory alpha-GC in the presence of NKT cells.

The alpha-GC compounds of this disclosure may be produced in vitro using a combinatorial approach, as described in the Examples. Briefly, this approach involves the generation of acyl chain building blocks and sphingoids having different terminal structures such as isomethyl (omega-2) and anteisomethyl (omega-3), and normal chains. The acyl building blocks are produced by (1) synthesizing (or providing) C13 alkyl iodide having diol at 1,3 position from L-(−)-malic acid, and (2) introducing an unbranched or branched structure, for example via sp³-sp³ cross-coupling, to the C13 alkyl iodide, followed by oxidation, to form acyl building blocks in the form of carboxylic acids. The sphinganine building blocks are produced by (1) preparing (or providing) alpha-galactosylsphinganine having iodide, for example from Garner aldehyde, (2) introducing an unbranched or branched structure, for example via sp³-sp³ cross-coupling, to form sphinganine building blocks. Once prepared, the acyl building blocks are allowed to react with the sphingoid building blocks to form an amide bond therebetween, thereby rendering all possible analogs with respect to chain lengths and structural diversity particularly at the terminal branches.

In another aspect, the invention provides a compound of formula (I):

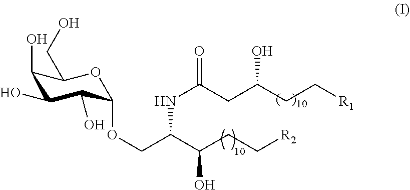

or an enantiomer or diastereomer thereof, wherein, R₁ is methyl, ethyl, or branched or unbranched C₃-C₆ alkyl and R₂ is methyl or branched C₃-C₆ alkyl. R₁ and R₂ are independently selected from these combinations.

In certain embodiments, R₁ and R₂ independently are selected from the group consisting of methyl, ethyl, n-propyl, isopropyl, n-butyl, sec-butyl, and n-pentyl. In certain embodiments, R₁ is selected from methyl, ethyl, isopropyl, and sec-butyl; and R₂ is selected from isopropyl, n-butyl, sec-butyl, and n-pentyl.

In certain embodiments, both of R₁ and R₂ are branched. In certain embodiments, R₂ is branched. In certain embodiments, R₁ is unbranched and R₂ is branched.

In certain embodiments, the compound of formula (I) is an isolated compound. In certain embodiments, the compound is substantially free of homologues, positional isomers, and stereoisomers. In certain embodiments, the compound is at least 50%, at least 60%, at least 70%, at least 80%, at least 90%, or at least 95% pure. Purity may be determined using routine methods in the art, such as HPLC in conjunction with a suitable detector (e.g., mass spectrometer)

### Synthesis

In another aspect, the invention provides method of making a compound of formula (I), comprising:

(1) condensing a compound of formula (II):

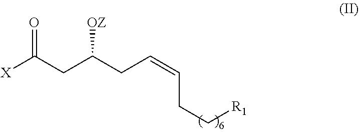

wherein X is —OH or a leaving group; and Z is an oxygen protecting group;
with a compound of formula (III):

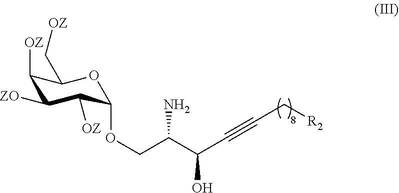

wherein each Z independently is an oxygen protecting group;
to afford a compound of formula (IV):

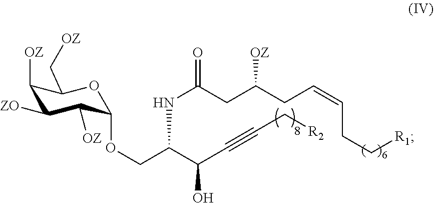

and

(2) reducing the unsaturated carbon-carbon bonds and deprotecting the protected hydroxyl groups to afford the compound of formula (I).

In certain embodiments of the method, X is —OH. In certain embodiments, X is a leaving group. As used herein, a “leaving group” is an art-understood term referring to a molecular fragment that departs with a pair of electrons in heterolytic bond cleavage, wherein the molecular fragment is an anion or neutral molecule. See, for example, Smith, March Advanced Organic Chemistry 6th ed. (501-502). Exemplary leaving groups include, but are not limited to, halo (e.g., chloro, bromo, iodo), carboxyl (e.g., acetate and benzoate) and sulfonyl substituted hydroxyl groups (e.g., tosyl, mesyl, besyl).

Oxygen protecting groups are well known in the art and include those described in detail in Protecting Groups in Organic Synthesis, T. W. Greene and P. G. M. Wuts, 3rd edition, John Wiley & Sons, 1999, incorporated herein by reference. Exemplary oxygen protecting groups include, but are not limited to, methyl, methoxylmethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl (Bn), p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, α-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodisulfuran-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), alkyl methyl carbonate, 9-fluorenylmethyl carbonate (Fmoc), alkyl ethyl carbonate, alkyl 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio) ethyl carbonate (Peoc), alkyl isobutyl carbonate, alkyl vinyl carbonate alkyl allyl carbonate, alkyl p-nitrophenyl carbonate, alkyl benzyl carbonate, alkyl p-methoxybenzyl carbonate, alkyl 3,4-dimethoxybenzyl carbonate, alkyl o-nitrobenzyl carbonate, alkyl p-nitrobenzyl carbonate, alkyl S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxyacyl)benzoate, α-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts).

In certain embodiments, Z is a protecting group that is cleavable by hydrogenolysis. In certain embodiments, Z is a benzyl group, optionally substituted with 1-3 C₁-C₆ alkoxy groups. In certain embodiments, each Z is benzyl (Bn).

In certain embodiments, step (1) comprises contacting the compound of formula (II) with the compound of formula (III) in the presence of a dehydration agent. The dehydration agent may be a carbodiimide such as DCC or EDC. In a particular embodiment, the dehydration agent is EDC.

In certain embodiments, step (2) comprises contacting the compound of formula (IV) with H₂ in the presence of a catalyst. The catalyst may comprise a transition metal such as palladium (Pd), platinum (Pt), rhodium (Rh), or iridium (Ir). In a particular embodiment, the catalyst is Pd(OH)₂.

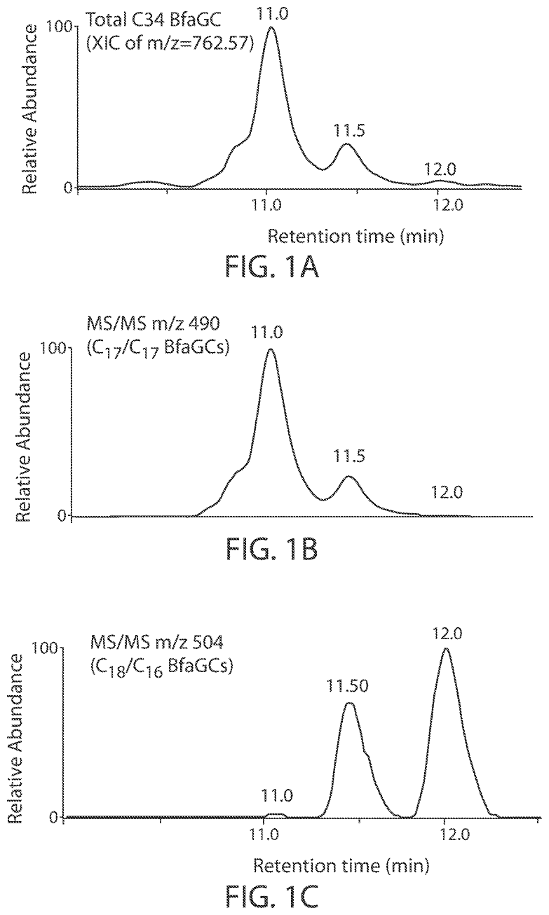

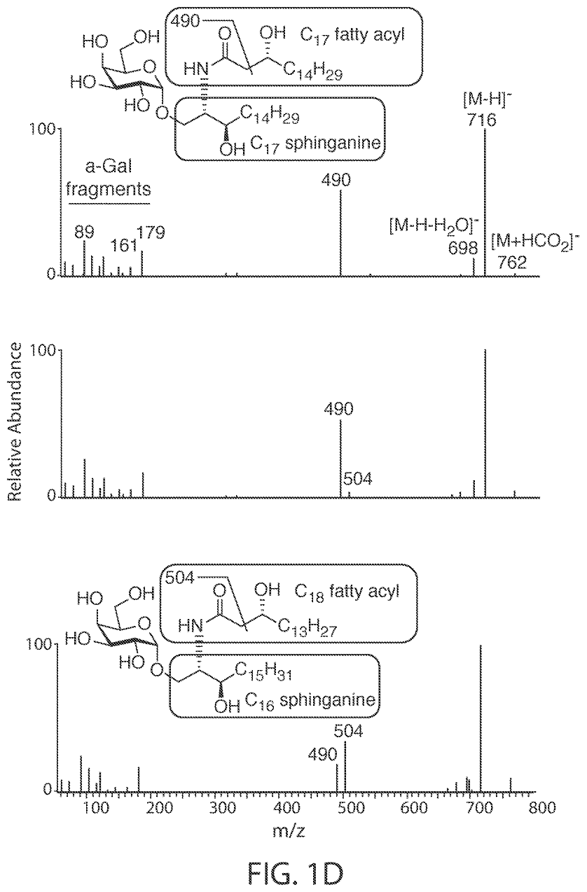

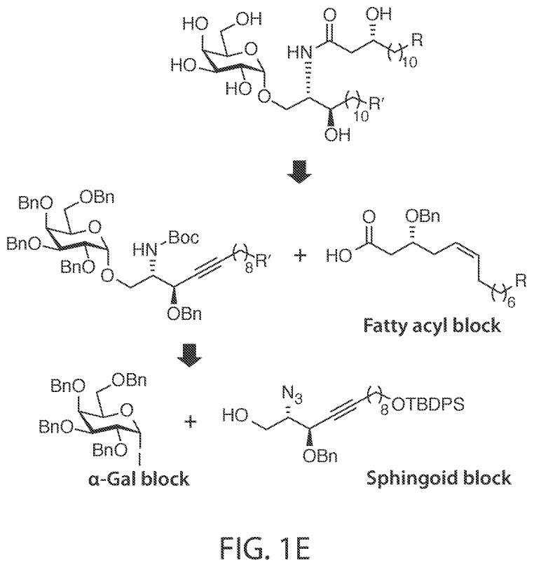

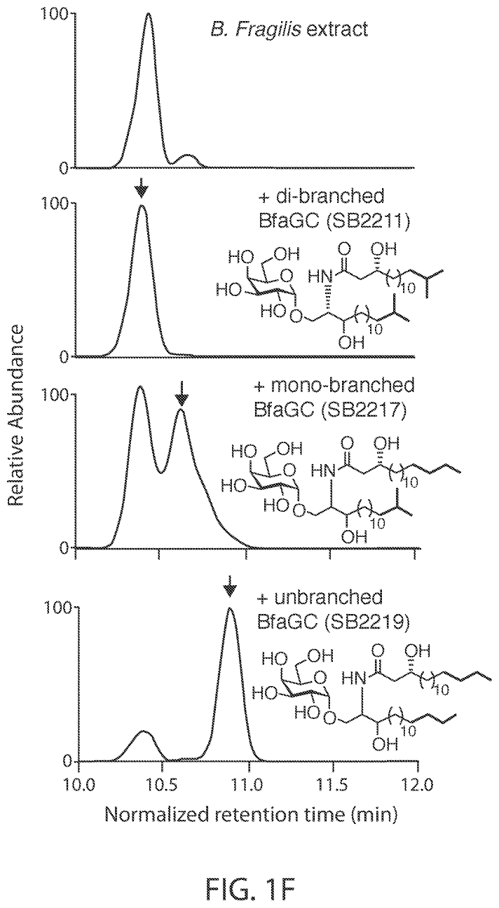

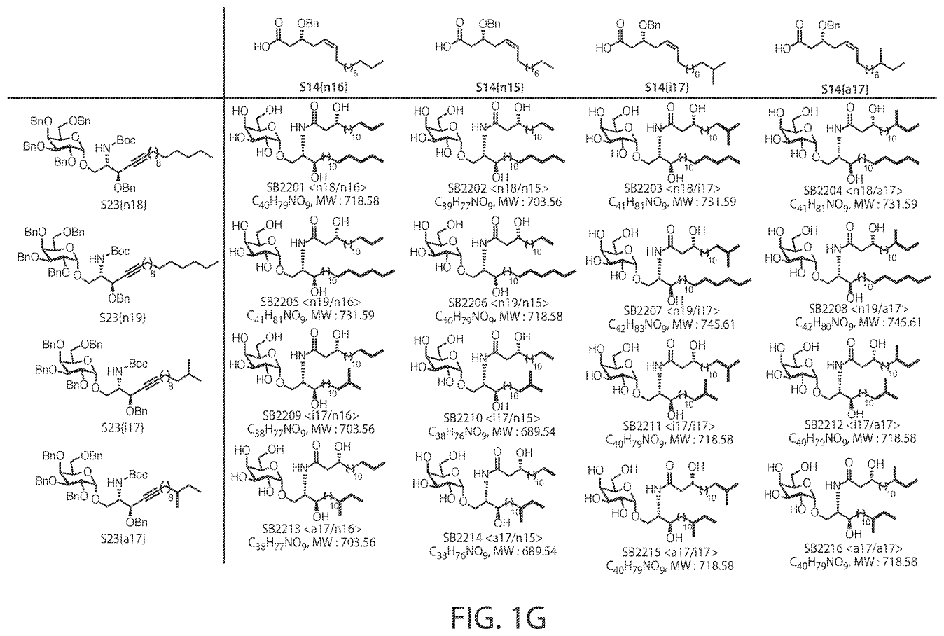

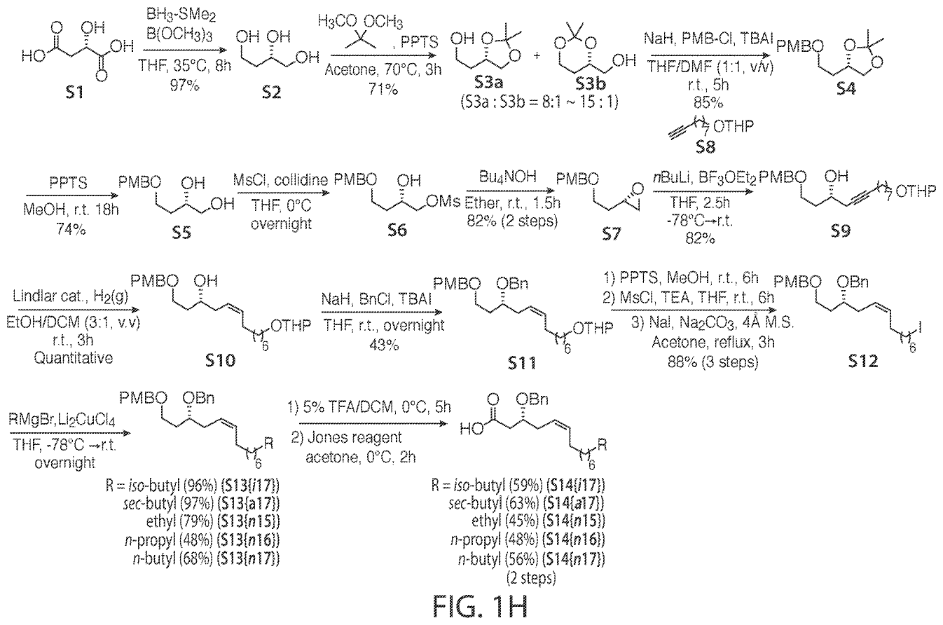

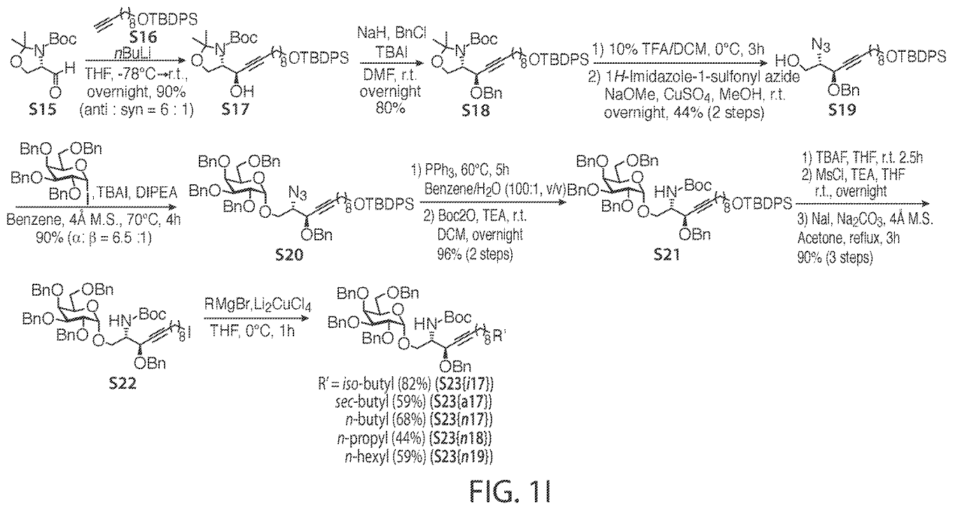

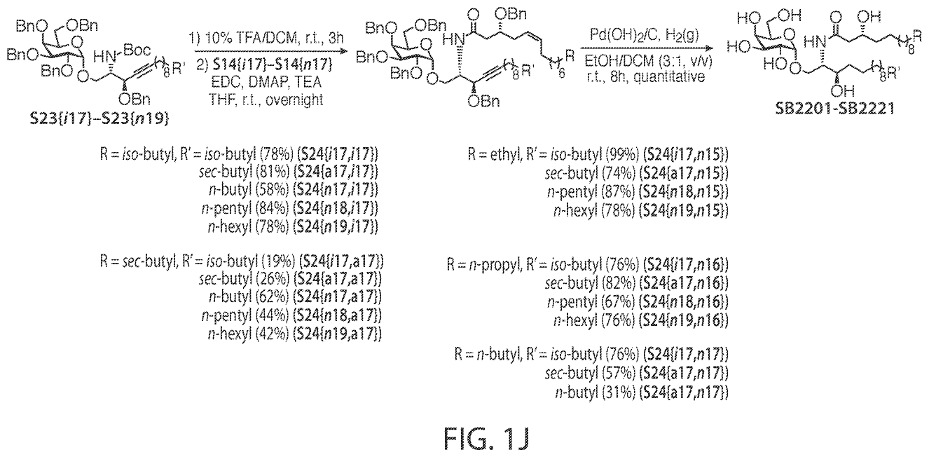

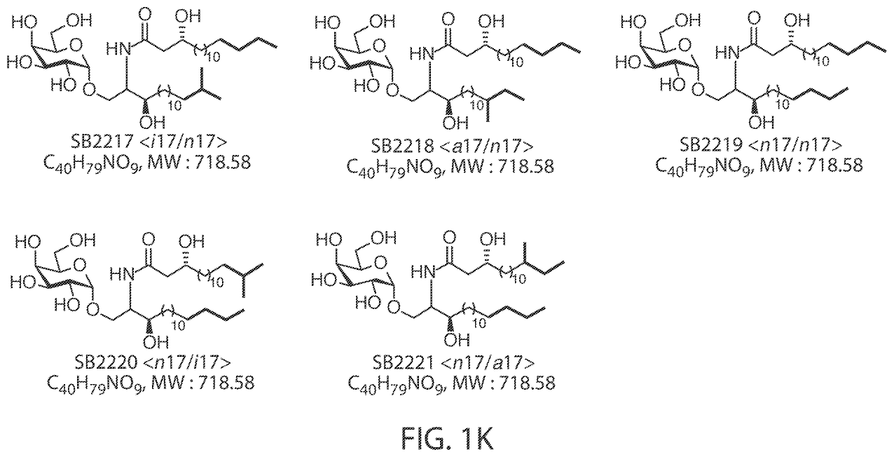

In certain embodiments, the method comprises one or more conditions of FIG. 1J.

In certain embodiments, the compound of formula (II) is prepared by a method comprising one or more conditions of FIG. 1H. In certain embodiments, the compound of formula (III) is prepared by a method comprising one or more conditions of FIG. 1I.

In certain embodiments, the method makes a plurality of compounds of formula (I), and comprises:

(1) condensing a plurality of compounds of formula (II) with a plurality of compounds of formula (III), to afford a plurality of compounds of formula (IV); and

(2) reducing the unsaturated carbon-carbon bonds and deprotecting the protected hydroxyl groups of the plurality of compounds of formula (IV) to afford the plurality of compounds of formula (I).

### Uses—In Vitro and In Vivo

The invention contemplates in vitro and in vivo uses of the alpha-GC and compositions provided herein. When used in vivo, the alpha-GC and compositions may be formulated as pharmaceutical compositions (or preparations), intending that they are suitable for administration to a subject. A pharmaceutical composition need not be therapeutic or prophylactic however (i.e., it may not eradicate an existing condition or prevent a condition from ever occurring in a subject). Instead, it may be used to modulate an aberrant immune response such as an increased NKT cell based immune response. The modulation of such aberrant immune response may be evidenced by a modulation of one or more symptoms resulting from the underlying NKT cell based immune response. It is to be understood however that the compounds provided herein impact the underlying condition and not simply one or more symptoms. Such in vivo uses may be in subjects being treated for a particular condition characterized by increased NKT cell number and/or NKT cell activity with the intention of providing some therapeutic or prophylactic benefit. Alternatively, such the compounds and compositions may be used in vivo for research purposes, inter alia, typically in non-human subjects. The compounds and compositions may be used in vitro to modulate immune responses involving activated NKT cells. Whether in vivo or in vitro, the compounds and compositions may be used in screening assays to identify NKT cell stimulatory agents or inhibitory agents.

Whether in vivo or in vitro, the compounds and compositions may be used in a method that involves contacting the compound or composition with an antigen presenting cell, and contacting the “loaded” antigen presenting cell with an NKT cell(s). The antigen presenting cells typically will express CD1d on their surface. A “loaded” antigen presenting cell intends an antigen presenting cell that has an immunoinhibitory alpha-GC molecule of the invention bound to its CD1d and is therefore able to present such molecule to an NKT cell. The contacting may occur in the presence of an agent that stimulates NKT cells such as the alpha-GC KRN7000. The contacting may occur in the absence of such an immunostimulatory agent, and instead the NKT cells may be activated NKT cells. Such cells may have been activated, for example in vitro, prior to the contacting step or they may have been obtained from a subject having an increased NKT cell based immune response. It is to be understood that these methods may be carried out in vivo or in vitro.

### Conditions

The alpha-GC and compositions thereof may be used to treat conditions that are characterized by increased levels of NKT cells and/or activity. An increased level of NKT cells or activity is measured relative to a normal subject (or a normal population of subjects) not having an inflammatory condition and nor at increased (or elevated, intending above-normal) risk of developing such a condition (e.g., as may be the case if the condition is inherited). NKT cells and/or activity may be measured in a blood sample or a biopsy such as a colonic (e.g., lamina propria) biopsy. Serum levels of proinflammatory cytokines, such as IFN-gamma, may be measured from the blood sample, for example. In some instances, persons having a family medical history or a personal medical history of a condition characterized by increased levels of NKT cells and/or activity, such as for example colitis, arthritis, asthma, and the like, may be presumed to have or be at risk of developing the condition even if they are not experiencing symptoms at or near the time of treatment. In some instances, the subject may have an allergy or an allergic disorder. In some instances, activated NKT cells are identified by the presence of CD1d tetramers, and may be detected and/or measured using for example flow cytometry or other immunostaining methods.

Such conditions include inflammatory conditions. Inflammatory conditions are conditions caused by, resulting from, or resulting in inflammation. An inflammatory condition may also refer to a dysregulated inflammatory reaction that causes an exaggerated response by macrophages, granulocytes, and/or T-lymphocytes leading to abnormal tissue damage and/or cell death. An inflammatory condition can be either an acute or chronic inflammatory condition and can result from infections or non-infectious causes. An inflammatory condition may be an autoimmune disease or it may be a non-autoimmune disease.

Inflammatory conditions include, without limitation, atherosclerosis, arteriosclerosis, polymyalgia rheumatica (PMR), gouty arthritis, degenerative arthritis, tendonitis, bursitis, psoriasis, cystic fibrosis, arthrosteitis, giant cell arteritis, polymyositis, dermatomyosis, pemphigus, pemphigoid, diabetes (e.g., Type I), myasthenia gravis, mixed connective tissue disease, sclerosing cholangitis, pernicious anemia, inflammatory dermatoses, usual interstitial pneumonitis (UIP), asbestosis, silicosis, bronchiectasis, berylliosis, talcosis, pneumoconiosis, sarcoidosis, desquamative interstitial pneumonia, lymphoid interstitial pneumonia, giant cell interstitial pneumonia, cellular interstitial pneumonia, extrinsic allergic alveolitis, angiitis (temporal arteritis and polyarteritis nodosa), inflammatory dermatoses, hepatitis, delayed-type hypersensitivity reactions (e.g., poison ivy dermatitis), pneumonia, respiratory tract inflammation, Adult Respiratory Distress Syndrome (ARDS), encephalitis, immediate hypersensitivity reactions, asthma, hayfever, allergies, acute anaphylaxis, rheumatic fever, pyelonephritis, cellulitis, cystitis, chronic cholecystitis, ischemia (ischemic injury), reperfusion injury, allograft rejection, host-versus-graft rejection, appendicitis, arteritis, blepharitis, bronchiolitis, bronchitis, cervicitis, cholangitis, chorioamnionitis, conjunctivitis, dacryoadenitis, dermatomyositis, endocarditis, endometritis, enteritis, enterocolitis, epicondylitis, epididymitis, fasciitis, fibrositis, gastritis, gastroenteritis, gingivitis, ileitis, iritis, laryngitis, myelitis, myocarditis, nephritis, omphalitis, oophoritis, orchitis, osteitis, otitis, pancreatitis, parotitis, pericarditis, pharyngitis, pleuritis, phlebitis, pneumonitis, proctitis, prostatitis, rhinitis, salpingitis, sinusitis, stomatitis, synovitis, testitis, tonsillitis, urethritis, urocystitis, uveitis, vaginitis, vasculitis, vulvitis, vulvovaginitis, angitis, chronic bronchitis, osteomylitis, optic neuritis, temporal arteritis, transverse myelitis, necrotizing fascilitis, and necrotizing enterocolitis.

Conditions characterized by increased levels of NKT cells and/or activity may be autoimmune diseases. Autoimmune diseases are diseases arising from an inappropriate immune response of the body of a subject against substances and tissues normally present in the body. In other words, the immune system mistakes some part of the body as a pathogen and attacks its own cells. This may be restricted to certain organs (e.g., in autoimmune thyroiditis) or involve a particular tissue in different places (e.g., Goodpasture's disease which may affect the basement membrane in both the lung and kidney). The treatment of autoimmune diseases is typically with immunosuppression, e.g., medications that decrease the immune response.

Exemplary autoimmune diseases include, but are not limited to, multiple sclerosis, inflammatory bowel diseases such as colitis (e.g., ulcerative colitis), juvenile inflammatory bowel disease, Crohn's disease, and ileitis, glomerulonephritis, Goodpasture's disease or syndrome, Graves' disease, necrotizing vasculitis, lymphadenitis, peri-arteritis nodosa, systemic lupus erythematosis, rheumatoid, arthritis, psoriatic arthritis, psoriasis, ulcerative colitis, systemic sclerosis, dermatomyositis/polymyositis, anti-phospholipid antibody syndrome, scleroderma, perphigus vulgaris, ANCA-associated vasculitis (e.g., Wegener's granulomatosis, microscopic polyangiitis), urveitis, Sjogren's syndrome, Reiter's syndrome, ankylosing spondylitis, Lyme arthritis, GuillainBarre syndrome, Hashimoto's thyroiditis, and cardiomyopathy.

In some embodiments, the condition is inflammatory dermatitis.

In certain embodiments, the compounds and compositions provided herein are intended for the prevention and/or treatment of inflammatory bowel disease such as colitis, asthma and multiple sclerosis.

In some instances, exposure to the compounds or compositions provided herein may reduce the likelihood of a subject developing a conditions characterized by increased NKT cells or activity. For example, the compounds may be used prophylactically and possibly chronically in order to prevent development of such conditions. The efficacy of such methods may be assessed by measuring NKT cells or activity in the subject prior to administration (e.g., prior to first administration) and following administration. The compound may be administered to the subject chronically, for example, every day, every week, every month, every several months, every year, etc.

The term “treat”, “treated,” “treating” or “treatment” is used herein to positively impact the condition characterized by increased NKT cell numbers and/or activity. Positive impact on the condition may be reflected by the ability to relieve, reduce or alleviate at least one symptom of the condition. For example, in the context of inflammatory bowel disease such as colitis, treatment may be evidenced by a maintenance or increase in subject weight. Treatment may also be reflected in a reduction in NKT cells and/or activity in the subject, including for example in the affected region of the subject (e.g., in the case of colitis, in the colon). Thus, treatment may be observed as diminishment of one or several symptoms of such a condition or as complete eradication of the condition. Within the meaning of the present invention, the term “treat” also denotes to arrest, delay the onset (i.e., the period prior to clinical manifestation of a condition) and/or reduce the risk of developing or worsening a condition. The term “protect” is used herein to mean prevent delay or treat, or all, as appropriate, development or continuance or aggravation of a condition in a subject.

A “subject” to which administration is contemplated includes, but is not limited to, humans and other non-human animals including, for example, companion animals such as dogs, cats, domesticated pigs, ferrets, hamsters, and the like; primates such as cynomolgus monkeys, rhesus monkeys, and the like; and agricultural animals such as cattle, pigs, horses, sheep, goats, birds (e.g., chickens, ducks, geese, and/or turkeys), and the like. In important embodiments, the subject is a human subject.

The subject may be of any age ranging from newborn to elderly. In some important embodiments, the subject is a pediatric subject such as a neonate, infant, child or adolescent. In some embodiments, the subject was delivered by cesarean section and/or was not breast-fed. In such embodiments, the invention contemplates administering the alpha-GC in order to render the subject resistant to conditions characterized by increased NKT cell numbers or activity. As discussed herein, such conditions include but are not limited to asthma and autoimmune diseases such as but not limited to colitis (e.g., ulcerative colitis). Thus, the invention contemplates prophylactic treatment of a subject to prevent such conditions from manifesting. Accordingly, in some embodiments, the subject may be less than 10 years of age, less than 5 years of age, less than 1 year of age, less than 6 months of age, or less than 1 month of age. In some embodiments, the subject was delivered by cesarean section and/or was not or is not being breast-fed. The invention further contemplates administration of older subjects such as adults. The subject may be a pregnant subject or a female subject of child-bearing age, either of which may optionally be at increased risk of developing a condition characterized by increased NKT cell numbers and/or activity (e.g., an autoimmune disease, asthma, and the like). In so doing, the method may be used to treat a fetus as well. These latter embodiments are premised, at least in part, on the surprising finding that it is possible to impart resistance to offspring by administering alpha-GC to their mother during pregnancy. This finding, among others, suggest that NKT cell numbers are set early in life, thereby dictating whether a person is more likely or less likely to develop conditions characterized by increased NKT cell numbers and/or activity.

This disclosure further contemplates that subjects may be treated once, twice or more times, over a period of time. This period of time may be days, weeks, months, or years. As an example, the alpha-GC may be administered daily or weekly in a subject experiencing symptoms associated with a condition characterized by increased NKT cell numbers or activity, until such symptoms are reduced or eliminated. As another example, the agents may be administered one or more times in the early years of life of a subject and then may be administered again after several years, as a “boost” to the original administration. This latter administration schedule could be similar to that used in more traditional vaccination schemes.

Some embodiments of the invention involve treatment of subjects having asthma or treatment of subjects prior to the onset of asthma (e.g., children). A “subject having asthma” is a subject that has a disorder of the respiratory system characterized by inflammation, narrowing of the airways and increased reactivity of the airways to inhaled agents. Asthma is frequently, although not exclusively associated with atopic or allergic symptoms. An “initiator” as used herein refers to a composition or environmental condition which triggers asthma. Initiators include, but are not limited to, allergens, cold temperatures, exercise, viral infections, and the like.

### Additional Active Agents

This disclosure contemplates the administration of alpha-GC with one or more additional active agents. The additional active agents include but are not limited to immunosuppressants or anti-inflammatory agents, asthma medicaments, allergy medicaments, and the like. The additional active agents may be blocking antibodies although they are not so limited. These second agents may be those that are used in the treatment or prevention of inflammatory conditions including autoimmune disorders such as but not limited to colitis.

Immunosuppressants or anti-inflammatory agents are agents that suppress or reduce an immune response. General classes of anti-inflammatories include steroids, non-steroid anti-inflammatory drugs (NSAIDS), as well as various classes listed herein.

Non-limiting examples of immunosuppressants or anti-inflammatory agents include without limitation Alclofenac; Alclometasone Dipropionate; Algestone Acetonide; Alpha Amylase; Amcinafal; Amcinafide; Amfenac Sodium; Amiprilose Hydrochloride; Anakinra; Anirolac; Anitrazafen; Apazone; Balsalazide Disodium; Bendazac; Benoxaprofen; Benzydamine Hydrochloride; Bromelains; Broperamole; Budesonide; Carprofen; Cicloprofen; Cintazone; Cliprofen; Clobetasol Propionate; Clobetasone Butyrate; Clopirac; Cloticasone Propionate; Cormethasone Acetate; Cortodoxone; Deflazacort; Desonide; Desoximetasone; Dexamethasone Dipropionate; Diclofenac Potassium; Diclofenac Sodium; Diflorasone Diacetate; Diflumidone Sodium; Diflunisal; Difluprednate; Diftalone; Dimethyl Sulfoxide; Drocinonide; Endrysone; Enlimomab; Enolicam Sodium; Epirizole; Etodolac; Etofenamate; Felbinac; Fenamole; Fenbufen; Fenclofenac; Fenclorac; Fendosal; Fenpipalone; Fentiazac; Flazalone; Fluazacort; Flufenamic Acid; Flumizole; Flunisolide Acetate; Flunixin; Flunixin Meglumine; Fluocortin Butyl; Fluorometholone Acetate; Fluquazone; Flurbiprofen; Fluretofen; Fluticasone Propionate; Furaprofen; Furobufen; Halcinonide; Halobetasol Propionate; Halopredone Acetate; Ibufenac; Ibuprofen; Ibuprofen Aluminum; Ibuprofen Piconol; Ilonidap; Indomethacin; Indomethacin Sodium; Indoprofen; Indoxole; Intrazole; Isoflupredone Acetate; Isoxepac; Isoxicam; Ketoprofen; Lofemizole Hydrochloride; Lornoxicam; Loteprednol Etabonate; Meclofenamate Sodium; Meclofenamic Acid; Meclorisone Dibutyrate; Mefenamic Acid; Mesalamine; Meseclazone; Methylprednisolone Suleptanate; Morniflumate; Nabumetone; Naproxen; Naproxen Sodium; Naproxol; Nimazone; Olsalazine Sodium; Orgotein; Orpanoxin; Oxaprozin; Oxyphenbutazone; Paranyline Hydrochloride; Pentosan Polysulfate Sodium; Phenbutazone Sodium Glycerate; Pirfenidone; Piroxicam; Piroxicam Cinnamate; Piroxicam Olamine; Pirprofen; Prednazate; Prifelone; Prodolic Acid; Proquazone; Proxazole; Proxazole Citrate; Rimexolone; Romazarit; Salcolex; Salnacedin; Salsalate; Salycilates; Sanguinarium Chloride; Seclazone; Sermetacin; Sudoxicam; Sulindac; Suprofen; Talmetacin; Talniflumate; Talosalate; Tebufelone; Tenidap; Tenidap Sodium; Tenoxicam; Tesicam; Tesimide; Tetrydamine; Tiopinac; Tixocortol Pivalate; Tolmetin; Tolmetin Sodium; Triclonide; Triflumidate; Zidometacin; Glucocorticoids; Zomepirac Sodium.

Anti-inflammatory agents typically prescribed for autoimmune diseases include mesalamine.

The additional active agent may be an asthma medicament, meaning a medicament that reduces the symptoms, inhibits the asthmatic reaction, or prevents the development of an asthmatic reaction. Various types of medicaments for the treatment of asthma are described in the Guidelines For The Diagnosis and Management of Asthma, Expert Panel Report 2, NIH Publication No. 97/4051, Jul. 19, 1997, the entire contents of which are incorporated herein by reference. Asthma medicaments include, but are not limited, PDE-4 inhibitors, Bronchodilator/beta-2 agonists, K+ channel openers, VLA-4 antagonists, Neurokin antagonists, TXA2 synthesis inhibitors, Xanthanines, Arachidonic acid antagonists, 5 lipoxygenase inhibitors, Thromboxin A2 receptor antagonists, Thromboxane A2 antagonists, Inhibitor of 5-lipox activation proteins, and Protease inhibitors.

Bronchodilator/beta-2 agonists are a class of compounds which cause bronchodilation or smooth muscle relaxation. Bronchodilator/beta-2 agonists include, but are not limited to, salmeterol, salbutamol, albuterol, terbutaline, D2522/formoterol, fenoterol, bitolterol, pirbuerol methylxanthines and orciprenaline. Long-acting beta-2 agonists include, but are not limited to, salmeterol and albuterol. These compounds are usually used in combination with corticosteroids. Methylxanthines, including for instance theophylline, have been used for long-term control and prevention of symptoms. Short-acting beta-2 agonists include, but are not limited to, albuterol, bitolterol, pirbuterol, and terbutaline.

Allergy medicaments include, but are not limited to, anti-histamines, steroids, and prostaglandin inducers. Anti-histamines include, but are not limited to, loratidine, cetirizine, buclizine, ceterizine analogues, fexofenadine, terfenadine, desloratadine, norastemizole, epinastine, ebastine, ebastine, astemizole, levocabastine, azelastine, tranilast, terfenadine, mizolastine, betatastine, CS 560, and HSR 609. Prostaglandin inducers include, but are not limited to, S-5751. The steroids include, but are not limited to, beclomethasone, fluticasone, tramcinolone, budesonide, corticosteroids and budesonide.

Corticosteroids include, but are not limited to, beclomethasome dipropionate, budesonide, flunisolide, fluticaosone, propionate, and triamcinoone acetonide. Systemic corticosteroids include, but are not limited to, methylprednisolone, prednisolone and prednisone.

Immunomodulators include, but are not limited to, the group consisting of anti-inflammatory agents, leukotriene antagonists, IL-4 muteins, soluble IL-4 receptors, immunosuppressants (such as Tolerizing peptide vaccine), anti-IL-4 antibodies, IL-4 antagonists, anti-IL-5 antibodies, soluble IL-13 receptor-Fc fusion proteins, anti-IL-9 antibodies, CCR3 antagonists, CCR5 antagonists, VLA-4 inhibitors, and, and Downregulators of IgE.

Leukotriene modifiers include, but are not limited to, zafirlukast tablets and zileuton tablets. Zileuton tablets function as 5-lipoxygenase inhibitors.

Other immunomodulators include neuropeptides such as substance P that have been shown to have immunomodulating properties. Substance P is a neuropeptide first identified in 1931 by Von Euler and Gaddum and see Chang et al. 1971. Nature (London) New Biol. 232:86-87 (1971).

Another class of compounds is the down-regulators of IgE. These compounds include peptides or other molecules with the ability to bind to the IgE receptor and thereby prevent binding of antigen-specific IgE. Another type of downregulator of IgE is a monoclonal antibody directed against the IgE receptor-binding region of the human IgE molecule. Thus, one type of downregulator of IgE is an anti-IgE antibody or antibody fragment. Anti-IgE is being developed by Genentech. One of skill in the art could prepare functionally active antibody fragments of binding peptides which have the same function. Other types of IgE downregulators are polypeptides capable of blocking the binding of the IgE antibody to the Fc receptors on the cell surfaces and displacing IgE from binding sites upon which IgE is already bound.

These types of asthma medicaments are sometimes classified as long-term control medications or quick-relief medications. Long-term control medications include compounds such as corticosteroids (also referred to as glucocorticoids), methylprednisolone, prednisolone, prednisone, chromolyn sodium, nedocromil, long-acting beta2-agonists, methylxanthines, and leukotriene modifiers. Quick relief medications are useful for providing quick relief of symptoms arising from allergic or asthmatic responses. Quick relief medications include short-acting beta2 agonists, anticholinergics and systemic corticosteroids. Anticholinergics include, but are not limited to, ipratrapoium bromide.

When two or more agents are administered to a subject, these can be administered simultaneously (e.g., where they are pre-mixed and administered together), substantially simultaneously (e.g., where they are administered one after another in the time it would take a medical practitioner to administer two agents to a subject), or sequentially with a period of time lapsing between the administrations. The two or more agents can also be administered by the same route or by a different route. For example, the agents may be all administered by inhalation. As another example, one agent may be administered by injection and another may be administered by inhalation.

When used with the alpha-GC of this disclosure, such second agents may be administered in a reduced dose. This may in turn reduce any severity or side effect observed with the second agent. The dose of the second agent may be reduced by 5%, 10%, 15%, 20%, 25%, 30%, 35%, 40%, 45%, 50%, or more. Alternatively or additionally, the second agent may be administered less frequently to the subject that is also receiving the alpha-GC of this disclosure. For example, the second agent may be administered at least 25% less frequently, at least 50% less frequently, or at least 75% less frequently.

### Pharmaceutical Compositions

The alpha-GC may be used (e.g., administered) in pharmaceutically acceptable preparations (or pharmaceutically acceptable compositions), typically when combined with a pharmaceutically acceptable carrier. Such preparations may routinely contain pharmaceutically acceptable concentrations of salt, buffering agents, preservatives, compatible carriers, and may optionally comprise other (i.e., secondary) therapeutic agents, as discussed above.

A pharmaceutically acceptable carrier is a pharmaceutically acceptable material, composition or vehicle, such as a liquid or solid filler, diluent, excipient, solvent or encapsulating material, involved in carrying or transporting a prophylactically or therapeutically active agent. Each carrier must be “acceptable” in the sense of being compatible with the other ingredients of the formulation and not injurious to the subject. Some examples of materials which can serve as pharmaceutically acceptable carriers include sugars, such as lactose, glucose and sucrose; glycols, such as propylene glycol; polyols, such as glycerin, sorbitol, mannitol and polyethylene glycol; esters, such as ethyl oleate and ethyl laurate; buffering agents, such as magnesium hydroxide and aluminum hydroxide; pyrogen-free water; isotonic saline; Ringer's solution; ethyl alcohol; phosphate buffer solutions; and other non-toxic compatible substances employed in pharmaceutical formulations.

The alpha-GC, when it is desirable to deliver them systemically, may be formulated for parenteral administration by injection, including for example by bolus injection or continuous infusion. Formulations for injection may be presented in unit dosage form, e.g., in ampoules or in multi-dose containers, with or without an added preservative.

The compositions may take such forms as water-soluble suspensions, solutions or emulsions in oily or aqueous vehicles, and may contain formulatory agents such as suspending, stabilizing and/or dispersing agents. Suitable lipophilic solvents or vehicles include fatty oils such as sesame oil, or synthetic fatty acid esters, such as ethyl oleate or triglycerides. Aqueous injection suspensions may contain substances which increase the viscosity of the suspension, such as sodium carboxymethyl cellulose, sorbitol, or dextran. Optionally, the suspension may also contain suitable stabilizers or agents which increase solubility. Alternatively, the agents may be in lyophilized or other powder or solid form for constitution with a suitable vehicle, e.g., sterile pyrogen-free water, before use.

Pharmaceutical compositions of the invention formulated for pulmonary delivery may provide the active ingredient in the form of droplets of a solution and/or suspension. Such formulations can be prepared, packaged, and/or sold as aqueous and/or dilute alcoholic solutions and/or suspensions, optionally sterile, comprising the active ingredient, and may conveniently be administered using any nebulization and/or atomization device. Such formulations may further comprise one or more additional ingredients including, but not limited to, a flavoring agent such as saccharin sodium, a volatile oil, a buffering agent, a surface active agent, and/or a preservative such as methylhydroxybenzoate. The droplets provided by this route of administration may have an average diameter in the range from about 0.1 to about 200 nanometers.

Formulations described herein as being useful for pulmonary delivery are useful for intranasal delivery of a pharmaceutical composition of the invention. Another formulation suitable for intranasal administration is a coarse powder comprising the active ingredient and having an average particle from about 0.2 to 500 micrometers. Such a formulation is administered by rapid inhalation through the nasal passage from a container of the powder held close to the nares.

Formulations for nasal administration may, for example, comprise from about as little as 0.1% (w/w) and as much as 100% (w/w) of the active ingredient, and may comprise one or more of the additional ingredients described herein. A pharmaceutical composition of the invention can be prepared, packaged, and/or sold in a formulation for buccal administration. Such formulations may, for example, be in the form of tablets and/or lozenges made using conventional methods, and may contain, for example, 0.1 to 20% (w/w) active ingredient, the balance comprising an orally dissolvable and/or degradable composition and, optionally, one or more of the additional ingredients described herein. Alternately, formulations for buccal administration may comprise a powder and/or an aerosolized and/or atomized solution and/or suspension comprising the active ingredient. Such powdered, aerosolized, and/or aerosolized formulations, when dispersed, may have an average particle and/or droplet size in the range from about 0.1 to about 200 nanometers, and may further comprise one or more of the additional ingredients described herein.

Other delivery systems can include time-release, delayed release or sustained release delivery systems. Such systems can avoid repeated administrations of the active compound, increasing convenience to the subject and the physician. Many types of release delivery systems are available and known to those of ordinary skill in the art. They include polymer base systems such as poly(lactide-glycolide), copolyoxalates, polycaprolactones, polyesteramides, polyorthoesters, polyhydroxybutyric acid, and polyanhydrides. Microcapsules of the foregoing polymers containing drugs are described in, for example, U.S. Pat. No. 5,075,109. Delivery systems also include non-polymer systems that are: lipids including sterols such as cholesterol, cholesterol esters and fatty acids or neutral fats such as mono- di- and tri-glycerides; hydrogel release systems; sylastic systems; peptide based systems; wax coatings; compressed tablets using conventional binders and excipients; partially fused implants; and the like. Specific examples include, but are not limited to: (a) erosional systems in which the active compound is contained in a form within a matrix such as those described in U.S. Pat. Nos. 4,452,775, 4,675,189 and 5,736,152, and (b) diffusional systems in which an active component permeates at a controlled rate from a polymer such as described in U.S. Pat. Nos. 3,854,480, 5,133,974 and 5,407,686. In addition, pump-based hardware delivery systems can be used, some of which are adapted for implantation.

Use of a long-term sustained release implant may be desirable. Long-term release, are used herein, means that the implant is constructed and arranged to delivery therapeutic levels of the active ingredient for at least 30 days, and preferably 60 days. Long-term sustained release implants are known to those of ordinary skill in the art and include some of the release systems described above.

The compounds provided herein may be administered to the gut through a number of different mechanisms. They may be formulated as oral formulations including oral solutions, suspensions, syrups, tincture, emulsions, elixirs, and the like. They may be formulated as capsules, including minicapsules, pills, tablets, including minitablets, lozenges, and the like. In some embodiments, these latter forms may be controlled release forms such that they release their contents in a controlled manner (e.g., after a certain period of time, for example after a certain period of time post-ingestion, or under a certain environment, for example in the gut). Controlled release forms include delayed release forms and extended release forms.

Strategies for targeting orally administered compounds to the gastrointestinal tract, including the gut, include covalent linkage of a drug with a carrier, including those that enhance stability as well as perhaps increase hydrophilicity; coating with pH-sensitive polymers; formulation of timed-release systems, exploitation of carriers that are degraded specifically by colonic bacteria; bioadhesive systems; and osmotic controlled drug delivery systems. Microbially degradable polymers especially azo crosslinked polymers may be used for targeting compounds to the colon. Certain plant polysaccharides such as amylose, inulin, pectin and guar gum which remain unaffected in the presence of gastrointestinal enzymes may be used in colon-targeted formulations. Additionally, combinations of plant polysaccharides with crustacean extract, including chitosan or derivatives thereof, may be used in colonic delivery systems.

Commonly used pH-dependent coating polymers are methacrylic acid copolymers, commonly known as Eudragit™ (Registered trademark of Evonik AG, Darmstadt, Germany).

EUDRAGIT™ polymers (available from Evonik) are polymeric lacquer substances based on acrylates and/or methacrylates. A suitable polymers include EUDRAGIT™ RL, EUDRAGIT™ RS, EUDRAGIT™ L, EUDRAGIT™ S, and EUDRAGIT™ E.

The formulation may comprise a protectant such as a proteolytic enzyme inhibitor.

The formulation may comprise an adhesive entity such as a muco- or bio-adhesive.

Oral solid or semi-solid formulations such as tablets or capsules may prepared with pharmaceutically acceptable excipients such as binding agents (e.g., polyvinylpyrrolidone, hydroxypropylcellulose or hydroxypropylmethylcellulose); fillers (e.g., cornstarch, lactose, microcrystalline cellulose or calcium phosphate); lubricants (e.g., magnesium stearate, talc, or silica); disintegrants (e.g., sodium starch glycollate); and/or wetting agents (e.g., sodium lauryl sulphate). In some instances, the tablets may be coated using suitable methods and coating materials such as OPADRY™ film coating systems available from Colorcon, West Point, Pa. (e.g., OPADRY™ OY Type, OYC Type, Organic Enteric OY-P Type, Aqueous Enteric OY-A Type, OY-PM Type and OPADRY™ White, 32K18400).

Liquid oral formulations may be prepared with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl p-hydroxy benzoates or sorbic acid).

### Effective Amounts

The preparations of the invention are administered in effective amounts. An effective amount is that amount of an agent that alone stimulates the desired outcome. In some embodiments, the desired outcome is a decrease in the number and/or activity of NKT cells (e.g., activated NKT cells). In some embodiments, the desired outcome is a decrease or elimination of symptoms associated with a condition characterized by increased number and/or activity of NKT cells, optionally for an extended period of time.

The absolute amount will depend upon a variety of factors, including the material selected for administration, whether the administration is in single or multiple doses, and individual patient parameters including age, physical condition, size, weight, and the stage of the disease. These factors are well known to those of ordinary skill in the art and can be addressed with no more than routine experimentation.

The exact amount of a compound required to achieve an effective amount will vary from subject to subject, depending, for example, on species, age, and general condition of a subject, severity of the side effects or disorder, identity of the particular compound, mode of administration, and the like. The desired dosage can be delivered three times a day, two times a day, once a day, every other day, every third day, every week, every two weeks, every three weeks, or every four weeks. In certain embodiments, the desired dosage can be delivered using multiple administrations (e.g., two, three, four, five, six, seven, eight, nine, ten, eleven, twelve, thirteen, fourteen, or more administrations).

In certain embodiments, an effective amount of a molecule or a preparation for administration one or more times a day to a 70 kg adult human may comprise about 0.0001 mg to about 3000 mg, about 0.0001 mg to about 2000 mg, about 0.0001 mg to about 1000 mg, about 0.001 mg to about 1000 mg, about 0.01 mg to about 1000 mg, about 0.1 mg to about 1000 mg, about 1 mg to about 1000 mg, about 1 mg to about 100 mg, about 10 mg to about 1000 mg, or about 100 mg to about 1000 mg, of a molecule per unit dosage form.

In certain embodiments, the agents may be at dosage levels sufficient to deliver from about 0.001 mg/kg to about 100 mg/kg, from about 0.01 mg/kg to about 50 mg/kg, preferably from about 0.1 mg/kg to about 40 mg/kg, preferably from about 0.5 mg/kg to about 30 mg/kg, from about 0.01 mg/kg to about 10 mg/kg, from about 0.1 mg/kg to about 10 mg/kg, and more preferably from about 1 mg/kg to about 25 mg/kg, of subject body weight per day, one or more times a day, to obtain the desired therapeutic effect.

It will be appreciated that dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult.

### Administration Routes

The alpha-GC and compositions provided herein can be administered by any route, including enteral (e.g., oral), parenteral, intravenous, intramuscular, intra-arterial, intramedullary, intrathecal, subcutaneous, intraventricular, transdermal, intradermal, rectal, intravaginal, intraperitoneal, topical (as by powders, ointments, creams, and/or drops), mucosal, nasal, buccal, sublingual; by intratracheal instillation, bronchial instillation, and/or inhalation; and/or as an oral spray, nasal spray, and/or aerosol. Specifically contemplated routes are oral administration, intravenous administration (e.g., systemic intravenous injection), regional administration via blood and/or lymph supply, and/or direct administration to an affected site. In general, the most appropriate route of administration will depend upon a variety of factors including the nature of the agent (e.g., its stability in the environment of the gastrointestinal tract), and/or the condition of the subject (e.g., whether the subject is able to tolerate oral administration).

In some embodiments, the alpha-GC are administered by any route that effects delivery to the lungs. Systemic administration routes such as intravenous bolus injection or continuous infusion are suitable. More direct routes such as intranasal administration, intratracheal administration (e.g., via intubation), and inhalation (e.g., via an aerosol through the mouth or nose) are also contemplated by the invention and in some instances may be more appropriate particularly where rapid action is necessary. As used herein, an aerosol is a suspension of liquid dispersed as small particles in a gas, and it includes a fine mist or a spray containing such particles. As used herein, aerosolization is the process of producing of an aerosol by transforming a liquid suspension into small particles or droplets. This may be done using an aerosol delivery system such as a pressurized pack or a nebulizer. Nebulizers include air-jet (i.e., pneumatic), ultrasonic, and vibrating-mesh nebulizers, for example with the use of a suitable propellant such as but not limited to dichlorodifluoromethane, trichlorofluoromethane, dichlorotetrafluoroethane, carbon dioxide or other suitable gas. In addition to nebulizers, other devices for pulmonary delivery include but are not limited to metered dose inhalers (MDIs) and dry powder inhalers (DPIs). Capsules and cartridges of for example gelatin for use in an inhaler or insufflator may be formulated containing lyophilized agents and a suitable powder base such as lactose or starch.

In some embodiments, the alpha-GC are administered by any route that effects delivery to the colon or gut. Ingestible forms, whether liquid, solid, or semi-solid, and rectal forms, such as suppositories or rectal rinses, are some non-limiting examples.

### Kits

The invention also encompasses a packaged and labeled pharmaceutical product. This article of manufacture or kit includes the appropriate unit dosage form in an appropriate vessel or container such as a glass vial or plastic ampoule or other container that is hermetically sealed. Preferably, the article of manufacture or kit further comprises instructions on how to use including how to administer the pharmaceutical product. The instructions may further contain informational material that advises a medical practitioner, technician or subject on how to appropriately prevent or treat the disease or disorder in question. In other words, the article of manufacture includes instructions indicating or suggesting a dosing regimen for use including but not limited to actual doses, monitoring procedures, and other monitoring information.

In some embodiments, the unit dosage form should be suitable for rectal or oral delivery for example by suppository or ingestible. In some embodiments, the unit dosage form should be suitable for pulmonary delivery for example by aerosol.

As with any pharmaceutical product, the packaging material and container are designed to protect the stability of the product during storage and shipment.

The kits may include agents in sterile aqueous suspensions that may be used directly or may be diluted with normal saline for intravenous injection or use in a nebulizer, or dilution or combination with surfactant for intratracheal administration. The kits may therefore also contain the diluent solution or agent, such as saline or surfactant. The kit may also include a pulmonary delivery device such as a nebulizer or disposable components therefore such as the mouthpiece, nosepiece, or mask.

### EXAMPLES

The following Examples are meant for illustrative purposes, and are not meant to be exclusive or limiting.

### Example 1: Host Diet Directs Structure and Immunomodulatory Capacity of Gut Symbiotic Molecules

During its natural functions of digesting and absorbing nutrients, the gastrointestinal tract of mammals provides a rich niche for symbiotic microbes that permits colonization at a very high density (˜10¹¹ microbial cells per gram of colonic content) and with great diversity (up to 500 species) (1). The host's diet shapes the gut microbiota structure, and the diverse secondary metabolites produced by bacterial species in turn influence the host's physiology. For example, a high-fiber diet promotes expansion of bacterial species that are capable of using indigestible oligosaccharide (2). Diverse metabolites produced during oligosaccharide fermentation serve as important molecular mediators for the host, not only as an energy source for the gut epithelium (3) but also as regulators of host immunologic processes (4). Clearly, diet can exert selective co-evolutionary pressure on both the host and the host's symbiotic microbiota.

The complexity of this tripartite interaction of host, microbiota, and diet makes this one of the most interesting and challenging arenas in which to decipher the molecular mechanisms affecting host health and disease (5). Diet (6) and xenobiotics (7) as well as host endogenous factors (8) can be metabolized by the gut microbiota to generate bioactive mediators (secondary or specialized metabolites (9)). In contrast, it is less well defined how the host's diet (which is shared with the gut microbiota) directly impacts the specific structure and function of endobiotic mediators (primary metabolites) and how these molecules influence host physiology.

Unique glycosphingolipid molecules have previously been identified from the human gut symbiont Bacteroides fragilis (10, 11). Chemical analyses have structurally classified these lipids as alpha-galactosylceramides (aGCs). Several molecules in this chemical class serve as activators of natural killer T (NKT) cells (12). aGCs are loaded onto the MHC-like molecule CD1d on antigen-presenting cells (APCs) and activate NKT cells via recognition by the semi-invariant T cell receptor (13). Strikingly, unlike previously identified aGCs, those identified from B. fragilis (BfaGCs) perform NKT cell-antagonistic functions and suppress colonic NKT cell proliferation in vivo. Colonic NKT cells are regulated by the gut microbiota during development; germ-free mice have higher levels of colonic NKT cells than conventional mice (14). Unregulated NKT cell proliferation and consequent colitis susceptibility in germ-free animals can be corrected by conventionalization (co-housing with conventional mice) very early in life. Of considerable interest, monocolonization with B. fragilis or oral administration of biogenic BfaGCs can replicate the impact of colonization by the entire gut microbiota on the number of colonic NKT cells. This unique immunomodulatory impact of BfaGCs calls for a study of structure-activity relationships aimed at understanding host colonic NKT cell-regulatory functions and how they relate to this ubiquitous human gut symbiont.

Sphingolipidomic profiling of B. fragilis determined that BfaGCs are a mixture of homologous acyl chains (saturated C₁₅-C₁₉ in both sphinganine and fatty acyl groups). The prominent C34 BfaGC (m.w.=717.58) is shown in FIG. 1A as three isobars with different retention times at 11.0, 11.5 and 12.0 minutes. MS/MS extracted ion chromatograms (MS2-XICs) of m/z 490—the fingerprint fragment of the C₁₇ sphinganine/C₁₇ fatty acyl (C₁₇/C₁₇) BfaGC (FIG. 1B)—imply that these three peaks are constitutional isomers (i.e., the same chain lengths with differently branched acyl chains). In addition, MS²-XICs of m/z 504 reveal additional BfaGC isobars eluting at 11.5 and 12.0 minutes (FIG. 1C), of which the chain distribution is C₁₈ sphinganine/C₁₆ fatty acyl (C₁₈/C₁₆). MS/MS spectra of three peaks (FIG. 1D) further confirm that C₁₇/C₁₇ and C₁₈/C₁₆ BfaGCs co-elute, showing that both m/z 490 and m/z 504 fragments coexist in the 11.5- and 12.0-minute peaks. Isobaric/isomeric mixtures are also identified in other BfaGCs with total carbon numbers of C₃₂-C₃₆.

Since the shape and the length of lipid chains are crucial components of CD1d-mediated NKT cell activation by aGCs, a total synthesis of BfaGC analogues with all possible chain lengths and terminal branches in both fatty acyl chains (blue block with R group) and sphingoid chains (red block with R′ group) was designed and pursued (FIG. 1E). This strategy allows comprehensive and unambiguous investigation of structural complexity. Sixteen BfaGC analogues (SB2201-SB2216, FIG. 1G), were systematically constructed using a matrix-based approach with acyl and sphingoid building blocks containing isomethyl (ω-2) and anteisomethyl (ω-3) branches (FIGS. 1H to 1J). To cover structural variants of prominent C34 BfaGCs, five constitutional isomers with different branched structures (SB2217-SB2221, FIG. 1K) were further synthesized. In total, 21 chain-length (C₁₅-C₁₉) and terminal branching BfaGC isomers were prepared (Table 1).

TABLE 1
List of 21 synthesized BfaGCs, SB2201-SB2221.

| Name | Molecular Weight | Sphingosine structure | Fatty acyl structure |
|---|---|---|---|
| SB2201 | 717.58 | n18 | n16 |
| SB2202 | 703.56 | n18 | n15 |
| SB2203 | 731.59 | n18 | i17 |
| SB2204 | 731.59 | n18 | a17 |
| SB2205 | 731.59 | n19 | n16 |
| SB2206 | 717.58 | n19 | n15 |
| SB2207 | 745.61 | n19 | i17 |
| SB2208 | 745.61 | n19 | a17 |
| SB2209 | 703.56 | i17 | n16 |
| SB2210 | 689.54 | i17 | n15 |
| SB2211 | 717.58 | i17 | i17 |
| SB2212 | 717.58 | i17 | a17 |
| SB2213 | 703.56 | a17 | n16 |
| SB2214 | 689.54 | a17 | n15 |
| SB2215 | 717.58 | a17 | i17 |
| SB2216 | 717.58 | a17 | a17 |
| SB2217 | 717.58 | i17 | n17 |
| SB2218 | 717.58 | a17 | n17 |
| SB2219 | 717.58 | n17 | n17 |
| SB2220 | 717.58 | n17 | i17 |
| SB2221 | 717.58 | n17 | a17 |

n straight-chain
i isomethyl(omega-2)-branching
a anteisomethyl(omega-3)-branching

A co-injection study of C₁₇/C₁₇ BfaGC isomers and other synthetic standards revealed that the liquid chromatographic mobility of BfaGCs is positively correlated with the number of branches in acyl chains: di-branched BfaGCs (i.e., branched in both sphinganine and acyl chains) elute first, with subsequent elution of mono-branched and unbranched isomers (FIG. 1F). Di-branched isomers are the major BfaGC species—a finding consistent with previous reports on the acyl chain composition of B. fragilis fatty acids (15) and ceramides (16), which have shown that isomethyl (ω-2) and anteisomethyl (ω-3) branched fatty acids are prominent.

Lipidomic profiling of a wide range of human gut and oral bacteria (59 species) was then performed. Although targeted LCMS/MS analysis identified ceramides of bacterial-specific structures (such as C17:0-C17:0 dihydroceramide) from 26 different species, mostly from the order Bacteroidales (data not shown), alpha-galactosyl derivatives with the aforementioned bacterial ceramide backbone structure were exclusively identified from B. fragilis (data not shown). Furthermore, LC-MS/MS analysis of multiple strains of B. fragilis, including lab strains (NCTC9343, YCH46 and 638R) as well as clinical isolates also revealed that all tested B. fragilis strains synthesize BfaGCs as well as ceramides and PE-ceramides (data not shown), confirming aGC biosynthesis is a uniquely conserved molecular phenotype of B. fragilis.

The finding that B. fragilis was the only species tested that made alpha-galactosylceramides led to a search for unique genes required for synthesis. Sphingolipid biosynthetic pathways in bacteria have been only partially characterized. While BF9343-2461 is annotated as an orthologue of eukaryotic serine palmytoyltransferase (SPT, 1,6) and SPT is well conserved among multiple gut and oral commensal species, downstream genes for ceramide and alpha-galactosylceramides synthesis have not been identified to date.

To overcome the very limited annotation information on BfaGC biosynthesis, a genome-wide, targeted metabolomics methodology was developed that can rapidly screen a large number of mutants and identify molecular phenotypes (data not shown). First, a trackable, random mutant library of B. fragilis was generated using mariner transposon insertion mutagenesis. Individual mutants were clonally expanded and targeted LC-MS/MS lipidomic analysis was carried out to identify mutants which cannot synthesize BfaGCs. Among 5580 mutants analyzed and 3458 verified to synthesize sphingolipids and cleared internal standard recovery cutoff, only ˜1% (total 35) transposon mutants had >90% decrease in C34 BfaGC:ceramide ratio (data not shown). Among them, we confirmed that one mutant (#8A7) nearly unable to synthesize BfaGCs (data not shown). Sequencing flanking regions of the transposon insert identified the gene as BF9343-3069. An in-frame deletion mutant was generated to confirm that when compared to wild-type B. fragilis, BF9343-A3069 show more than 98% decrease level of BfaGC, both when grown in culture or in the host gut but synthesizes an otherwise normal array of non-glycosylated sphingolipids (data now shown).

To assess whether BfaGCs are required for host colonic NKT cell regulation, mice monocolonized with BF9343-A3069 at birth were generated. These mice had significantly higher colonic NKT cells as adults than that of SPF mice or mice monocolonized at birth with wild-type B. fragilis (data not shown). As expected, BF9343-A3069 monocolonized animals were more susceptible to oxazolone-mediated colitis than wild-type B. fragilis monocolonized mice (data not shown). These results confirm that BfaGCs are necessary to modulate host colonic NKT cells by B. fragilis monoassociation.

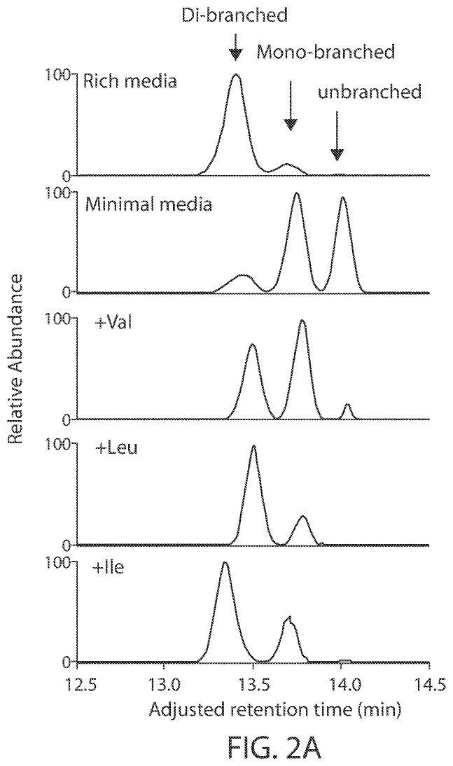

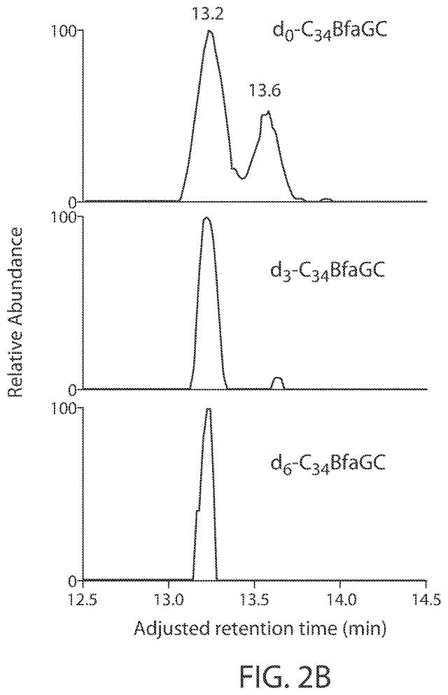

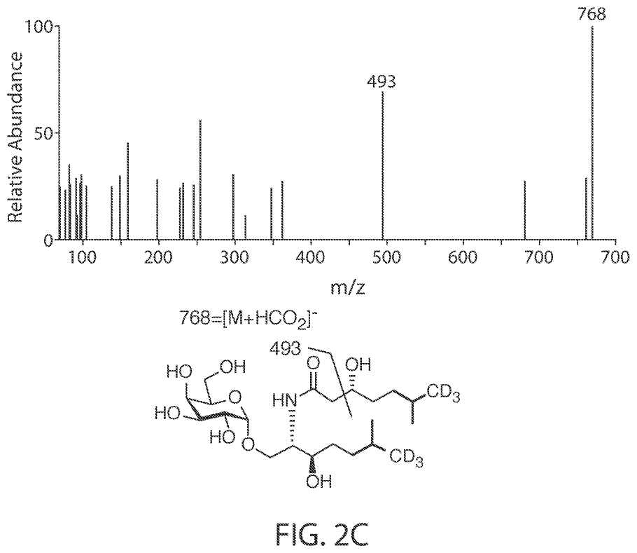

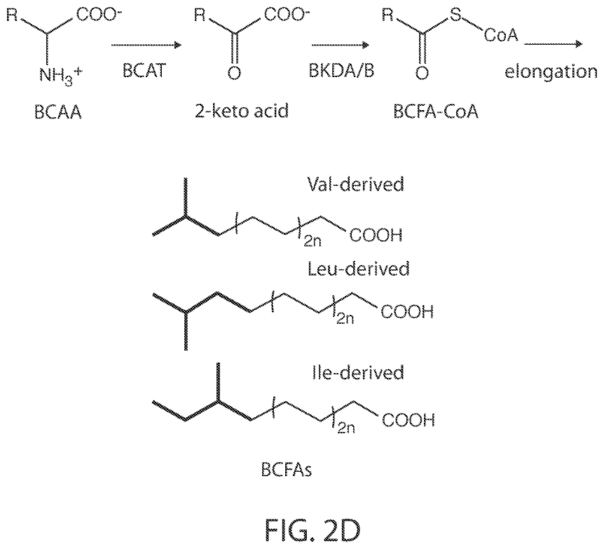

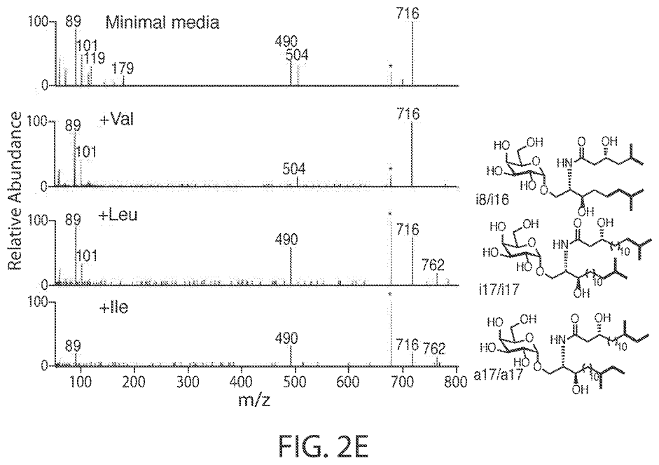

Odd-numbered and/or branched-chain fatty acids are relatively rare in eukaryotes but are more widely found in prokaryotes (17). In some bacterial species, the characteristic biosynthesis of these fatty acids involves the incorporation of deaminated branched-chain amino acids (BCAAs) such as valine, leucine, and isoleucine (18, 19). To assess the impact of exogenous BCAAs on BfaGC biosynthesis, branched BfaGC production was first investigated by limiting or supplementing exogenous BCAAs in vitro. When B. fragilis was grown in defined minimal medium (20) without amino acids, production of BfaGCs was skewed more toward unbranched lipids than it was in BCAA-rich medium. This skew could be reversed by supplementing the medium with BCAAs (FIG. 2A). Furthermore, LC-MS/MS analysis determined that supplementation with valine (which is converted to C4 (isobutyl)-CoA in vivo; FIG. 2D) produces C₁₈/C₁₆ BfaGCs as the major species, whereas supplementation with leucine or isoleucine (converted to C₅ (2- or 3-methyl-butyl)-CoA, respectively) produces C₁₇/C₁₇ BfaGCs (FIG. 2E). These results strongly implied that BCAAs dictate BfaGC structure by being directly incorporated into lipids. In vivo stable isotope tracking with deuterium-labeled leucine confirmed that host dietary BCAAs are incorporated into microbial sphingolipids, as shown by MS/MS fingerprinting of deuterium-incorporated acyl chain(s) of BfaGCs (FIGS. 2B and 2C). Furthermore, BCAA removal from the host's diet increases the ratio of mono-branched to di-branched sphingolipids, a change indicating the shift to straight-chain sphingolipids in BCAA-deprived medium in vivo (FIG. 2E). These results provide molecular-level evidence that host dietary components can dictate the structure of commensal molecules biosynthesized in the host's gut lumen.

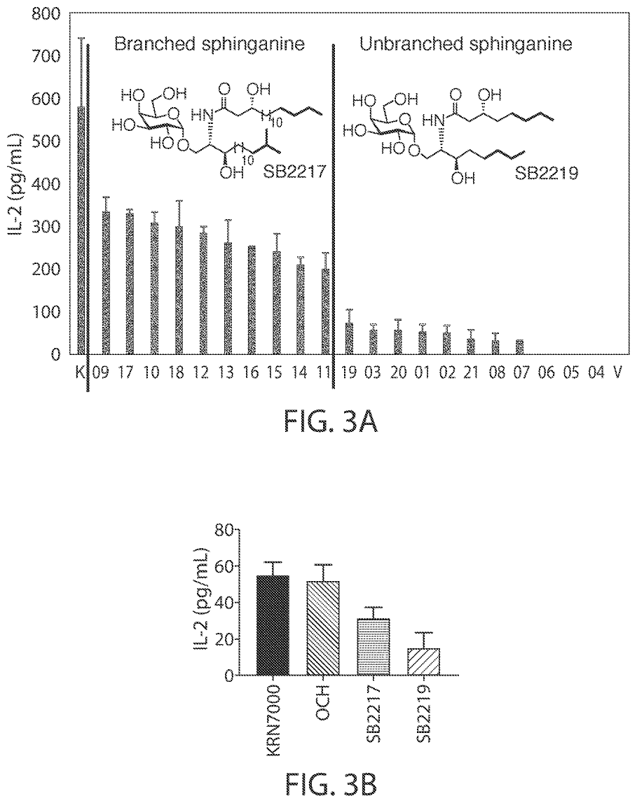

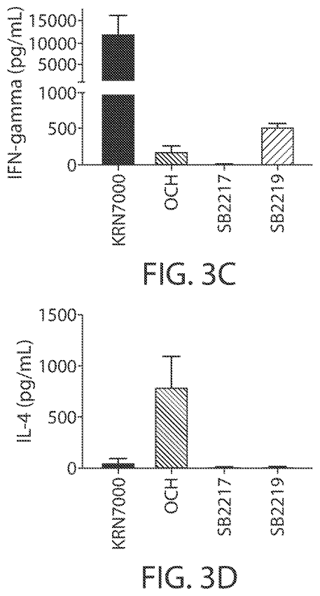

To investigate the relevance of structural variation to immunomodulatory function, an in vitro panel screening assay of synthetic BfaGCs was carried out in a co-culture system with bone marrow-derived dendritic cells (BMDCs) and NKT cells. Levels of interleukin 2 (IL-2) production in the presence of individual BfaGCs clearly distinguished two groups with different levels of NKT cell recognition: strong and weak stimulators (FIG. 3A). Strikingly, all 10 strong stimulators have branched (either ω-2 or ω-3) sphinganine chains, whereas none of 11 weak stimulators have such branching. In an assessment of how different levels of NKT cell responses in vitro correlate with NKT cell activity in vivo, a representative isobaric pair of molecules (FIG. 1K) from the strong stimulator group (SB2217: isomethyl branching in C₁₇ sphinganine) and the weak stimulator group (SB2219: no branching in C₁₇ sphinganine) were selected for in vivo experiments. These molecules were individually injected into mice (1 μg per mouse, intraperitoneally), and systemic levels of cytokines were quantified and directly compared to those in mice injected with KRN7000 (a prototypic Th1 agonist) and mice injected with OCH (a Th2 agonist). Serum IL-2 levels 2 h after injection show trends consistent with in vitro results (KRN7000>SB2217>SB2219), which implies that BfaGCs are also functioning in vivo (FIG. 3B). Surprisingly, when directly compared with KRN7000 and OCH, compound SB2219 only weakly induces a Th1 cytokine (interferon-γ) and does not induce Th2 cytokine IL-4, while compound SB2217 evokes neither interferon-γ (IFN-γ) nor IL-4 at the observed time points in vivo (FIGS. 3C and 3D, respectively). These results show that the efficacy of aGCs in producing IL-2 in APC-NKT cell co-culture is not always linearly related to Th1- or Th2-cytokine production in vivo. Although It is well established that aGCs with very long fatty acyl chains (e.g., KRN7000, which has a C26 saturated chain) selectively induce Th1-type cytokines and that shorter-sphingoid-chain aGCs (e.g., OCH, which has a C₈ saturated chain) skew toward Th2, aGCs with acyl and sphingoid chain lengths of long-chain fatty acids (C₁₅-C₁₉), prominently identified in B. fragilis, are less well investigated (21). Results of the in vitro co-culture and in vivo cytokine production experiments are consistent with the inhibition of cytokine production by biogenic BfaGCs competing against KRN7000(10), implying that BfaGCs—especially branched-chain BfaGCs—are recognized by NKT cells and may have previously unknown regulatory functions.

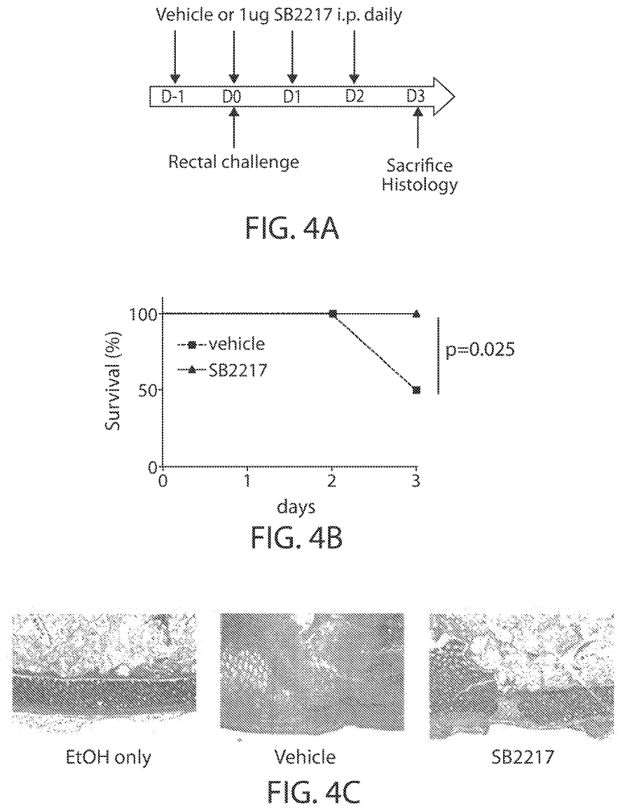

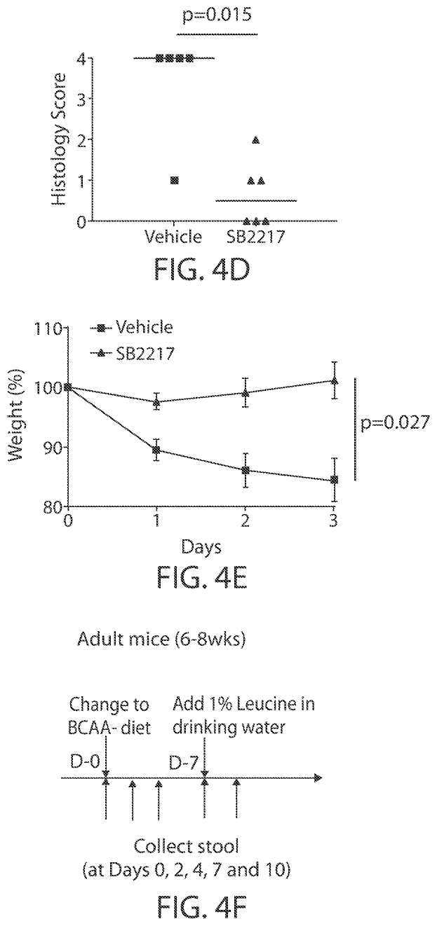

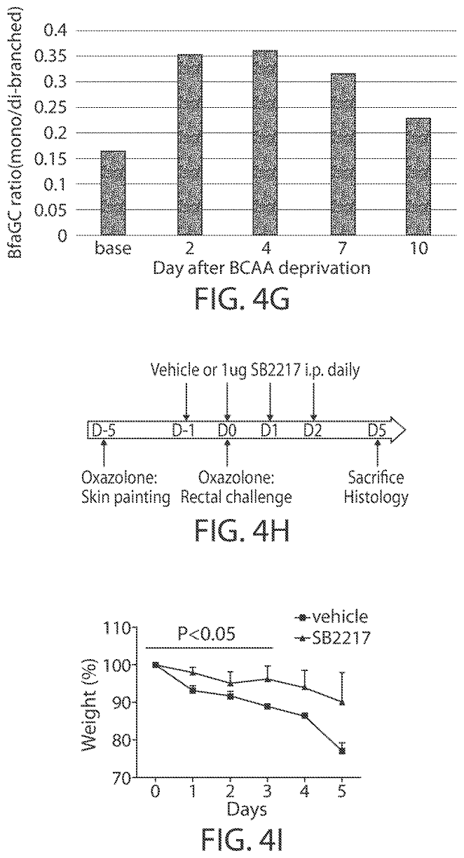

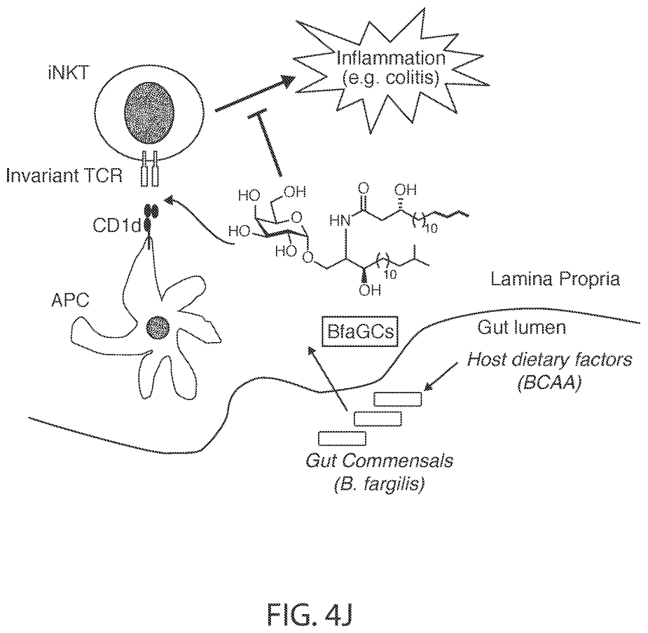

NKT cell development depends on the presence and composition of the microbiota early in life. Uncontrolled proliferation of colonic NKT cells, which causes susceptibility to NKT cell-mediated colitis in adulthood, can be rescued only by early colonization with a commensal microbiota (14) or B. fragilis or by administration of biogenic BfaGCs (10). However, this “window-of-opportunity” concept does not exclude the possibility of directly regulating NKT cell function with symbiotic molecules in normal or disease-prone conditions. To investigate this possibility, an NKT cell-driven oxazolone colitis model was used to assess host-protective actions of sphinganine-branched BfaGCs. Intraperitoneal administration of compound SB2217 protects wild-type animals from disease, reducing mortality, weight loss, and colonic inflammation (FIG. 4). Furthermore, SB2217 administration protects germ-free adult animals (FIGS. 4H and 4I), which have higher levels of colonic NKT cells and develop more severe colitis (14). These results show that branched BfaGCs can directly regulate NKT cells and protect the host, independent of basal colonic NKT cell levels or the colonization status of the host.

Canonical functions of NKT cells have been appreciated in the context of host defense and immunity, and aGCs have been recognized as potent agonists of NKT cell-mediated immune stimulation. Various synthetic aGCs have been investigated as anti-tumor therapeutics (22) and as vaccine adjuvants (23). On the other hand, recent results recognize NKT cell-mediated immunomodulatory actions (21). A subset of NKT cells is functionally characterized as anti-inflammatory (24), and NKT cells can promote IL-10 production by intestinal epithelial cells in a CD1d-dependent manner (25, 26). The present finding of host diet-directed, NKT cell-regulating BfaGCs with a unique structural moiety implies a novel mechanism of ligand-dependent immunomodulation of NKT cells. NKT ligand-mediated protection from colitis can be extended to other (DSS (27, 28) or TNBS (29)) colitis models. BfaGCs may be widely applicable therapeutic leads for targeting diverse NKT cell-mediated immune diseases, such as inflammatory bowel disease, asthma, and multiple sclerosis. Additional structural and pharmacologic studies may be performed to provide a structure-based explanation for NKT cell regulation.

Diet is a crucial factor in host health, and the gut microbiota clearly plays a critical role at this interface. Provided herein is molecular-level evidence that a specific dietary factor (the BCAA) directly impacts the structure and function of endobiotic immunomodulatory molecules (FIG. 4J). In contrast to passive metabolism of host- and diet-derived molecules, this is an active mechanism through which a gut symbiont participates in the host's immune maturation, producing its own mediators from host dietary factors.

### Materials and Methods

### Mice

All mice were 5-8 weeks old, and the experimental groups were age-matched with one another. Germ-free Swiss-Webster mice were bred and maintained in plastic isolators in the Harvard University animal facility. SPF Swiss-Webster mice were purchased from Taconic.

For in vivo isotope tracking and BCAA deprivation experiments, in-house bred, B. fragilis-monocolonized C57B6/L mice were used. Mice were treated outside the isolator, with the use of sterile technique during experimental period.

### Total Organic Synthesis of BfaGC Analogues

Materials.

All commercially available reagents were purchased from Sigma-Aldrich, Tokyo Chemical Industry Co., Ltd, or ThermoFisher Scientific and used without further purification unless otherwise specified. Solvents were purchased from commercial venders and used without further purification unless otherwise mentioned. Dry solvents were prepared using ultimate solvent purification system CT-SPS-SA [Glass Contour]. The progress of reaction was monitored using thin-layer chromatography (TLC) (silica gel 60, F₂₅₄ 0.25 mm). Components on TLC were visualized by treating the TLC plates with p-anisaldehyde, KMnO₄, or phosphomolybdic acid followed by heating. The compounds were purified by flash column chromatography on silica-gel (230-400 mesh). The eluent used for purification is reported in parentheses.

Compound Characterization.

The optical rotations were measured by JP/P-1030 [JASCO] using a sodium lamp (D line, 589 nm). ¹H and ¹³C NMR spectra were obtained on Bruker DRX-300 [Bruker Biospin], Agilent 400-MR DD2 [Agilent Technologies], or Varian Inova-500 [Varian Associates]. Chemical shifts were reported in parts per million (6, ppm). ¹H NMR spectra were calibrated using the residual solvent peak (CDCl₃ 7.26 ppm) or tetramethylsilane (TMS, 0.00 ppm) as internal standard. ¹³C NMR spectra were calibrated using the residual solvent peak (CDCl₃ 77.23 ppm; CD₃OD 49.00 ppm). Multiplicity was noted as: s (singlet); d (doublet); t (triplet); q (quartet); m (multiplet); dd (doublet of doublet); dt (doublet of triplet); td (triplet of doublet); br s (broad singlet), etc. Coupling constants were reported in Hz. Low resolution mass spectrometry (LRMS) was obtained by LCMS-2020 [Shimadzu] and LTQ [Thermo Fisher Scientific Inc.] using electron spray ionization (ESI).

Synthesis Strategies.

To maximize the synthetic efficacy and structural diversity, the synthetic route was designed as combinatorial approach between acyl building blocks and sphingoids having different terminal structures such as isomethyl (ω-2), anteisomethyl (ω-3) branches, and normal chains. For the preparation of acyl building blocks, C₁₃ alkyl iodide having diol at 1,3 position (S12 in FIG. 1H) was first synthesized from 1-(−)-malic acid. Then acyl building blocks (S14{i17}-S14{n16}) were obtained as a carboxylic acid form after the introduction of various normal and branch structure via sp³-sp³ cross-coupling followed by oxidation. In the case of sphinganine building blocks, α-galactosylsphinganine having iodide (S22 in FIG. 1I) was prepared from Garner aldehyde. In a similar manner as acyl building blocks, sphingoid building blocks (S23{i17}-S23{n18}) were generated by decoration of terminal structures via sp³-sp³ cross-coupling. After the preparation of acyl and sphingoid building blocks, the combining each building blocks with amide bond provided all possible BfaGC analogues (SB2201-SB2216) covering full mass ranges (C₃₂-C₃₆) as well as structural diversity at the terminal position of both acyl chain and sphingosine (FIG. 1J). Newly synthesized 16-member BfaGC analogue library covers a full mass range of major extracted BfaGC(C₃₂-C₃₆) along with excellent structural variation. By combining synthetic building blocks having iso-, anteiso-, or normal chain at the terminal position, no-, mono-, and di-branched BfaGC analogues were easily generated. In this context, five additional isobaric C34-BfaGC analogues (SB2217-SB2221, FIG. 1K) were synthesized to make a full coverage of structural diversity of C₁₇/C₁₇ isomers and a total 21-member BfaGC analogue library was completed to synthesis (Table 1).

### LC-MS/MS Lipidomics

An LC-MS/MS system (Thermo Scientific Vanquish RP-UPLC, connected to a Q Exactive Orbitrap) was used for sphingolipid profiling (10). An Agilent Zorbax C18 column (4.6×75 mm×5 μm) was used in the gradient LC elution condition (600 μL/min, 95% acetonitrile (ACN)/10 mM ammonium formate to 65% 2-propanol/30% ACN/10 mM ammonium formate) over 20 minutes. Data-dependent and parallel reaction monitoring with C₃₂-C₃₆ ceramides and BfaGCs or a data-dependent MS/MS method was used for analysis.

### Co-Injection (Matching) Study

Synthetic BfaGCs (SB2211, SB2217, SB2219) were individually spiked with a total lipid extract of cultured B. fragilis grown in rich medium.

### BCAA Incorporation/Tracking

Bacteria of B. fragilis strain NCTC 9343 were streaked onto a hemin agar plate and grown overnight. Colonies were inoculated onto rich medium first, and 10⁸ cells were then inoculated onto rich, defined, or BCAA-supplemented medium and grown to an OD of 0.5. Cells were harvested by centrifugation, and total lipid was extracted with MTBE-MeOH-water (30).

### In Vivo BCAA Manipulation

B. fragilis-monocolonized mice were given a 1% ²H₃-leucine solution as drinking water ad lib for 10 days. At days 3, 7, and 10, fecal pellets were collected and total lipids were extracted for LC-MS/MS analysis.

### LC-MS/MS Confirmation of Deuterium-Labeled BCAAs

A targeted LC-MS/MS method for unlabeled, ²H₃-C₃₄ ([M+HCO₂⁻]=765.6), and ²H₆-C34-BfaGCs ([M+HCO₂⁻]=768.6) was used to selectively identify BCAA-incorporated BfaGCs. Deuterium-incorporated species identified by extracted ion chromatography were further confirmed by retention time matching with a synthetic standard and MS/MS fragmentation shift by deuterium incorporation in the acyl chains.

### APC-NKT Co-Culture Assay for Panel Screening of Synthetic BfaGCs

IL-2 producing potential of synthetic BfaGCs were assessed with a slightly modified protocol which was described before¹. Briefly, 5×10⁴ BMDCs (bone-marrow monocytes cultured for 8 days in presence of GM-CSF) was pre-incubated with 300 nM individual BfaGCs. After 2 hours, 5×10⁴ 24.7 NKT hybridoma cells were added and incubated for 18 hours. Culture supernatants were analyzed by IL-2 ELISA (R&D Bioscience).

### In Vivo Cytokine Assay

Mice (n=4 per group) were given 100 uL of KRN7000, OCH, SB2217 or SB2219 solution (1 ug, 0.9% DMSO in PBS) intraperitoneally. Animals were bled at 2 hours after injection from tail vein (˜100 uL per animal). At 18 hours after injection, animals were euthanized and second 100 uL blood was drawn by cardiac puncture. Fresh blood was collected to Z-Gel tubes (Sarstedt) to form blood clot and spun at 10,000×g for 5 minutes for serum separation. Samples were frozen at −80 degree until analysis by multiplex ELISA (Eve Technologies, Calgary, Canada).

Oxazolone Colitis with BfaGC Treatment

Six to eight mice were used per each treatment group. From 1 day before until 2 days after rectal challenge, animals were given daily intraperitoneal doses (total, 4 doses) of vehicle or 1 μg of an SB2217 compound in 0.9% DMSO/PBS solution.

For SPF mice, a 3-day direct challenge model was used (26). On day 0, animals were given oxazolone intrarectally in 50% EtOH solution. Animals were monitored daily for weight loss. On day 3, mice were euthanized and their colons excised for histopathology scoring and cytokine production assay.

For germ-free mice, a 5-day pre-sensitization model was used. Five days before rectal challenge, oxazolone in 50% EtOH solution was painted onto the shaved back of the animals. On day 0, animals were given oxazolone intrarectally in 50% EtOH solution. Animals were monitored daily for weight loss. On day 5, mice were euthanized and their colons excised for histopathology scoring. Both colitis models were run three times and the representative among the results with the same trend was shown.

### Histopathology Scoring

A pathologist blinded to treatment groups conducted the histologic assessment of colons. The histologic score represented the combined scores for inflammation and ulceration; both elements were scored 0-4, with 0: normal, 1: limited loss of epithelium, 2: several lesions with epithelial loss, 3: epithelial loss with inflammation, and 4: severe inflammation and tissue necrosis.

### REFERENCES

1. The Human Microbiome Project Consortium, Structure, function and diversity of the healthy human microbiome. Nature. 486, 207-14 (2012).
2. G. D. Wu et al., Linking long-term dietary patterns with gut microbial enterotypes. Science. 334, 105-8 (2011).
3. G. den Besten et al., The role of short-chain fatty acids in the interplay between diet, gut microbiota, and host energy metabolism. J. Lipid Res. 54, 2325-40 (2013).
4. P. M. Smith et al., The Microbial Metabolites, Short-Chain Fatty Acids, Regulate Colonic Treg Cell Homeostasis. Science (80-.). 341, 569-573 (2013).
5. N. K. Surana, D. L. Kasper, Deciphering the tête-à-tête between the microbiota and the immune system. J. Clin. Invest. 124, 4197-203 (2014).
6. Z. Wang et al., Gut flora metabolism of phosphatidylcholine promotes cardiovascular disease. Nature. 472, 57-63 (2011).
7. R. N. Carmody, P. J. Turnbaugh, Host-microbial interactions in the metabolism of therapeutic and diet-derived xenobiotics. J. Clin. Invest. 124, 4173-81 (2014).
8. V. Tremaroli, F. Backhed, Functional interactions between the gut microbiota and host metabolism. Nature. 489, 242-249 (2012).
9. J. K. Nicholson et al., Host-gut microbiota metabolic interactions. Science. 336, 1262-7 (2012).
10. D. An et al., Sphingolipids from a Symbiotic Microbe Regulate Homeostasis of Host Intestinal Natural Killer T Cells. Cell. 156, 123-133 (2014).
11. L. C. Wieland Brown et al., Production of α-Galactosylceramide by a Prominent Member of the Human Gut Microbiota. PLoS Biol. 11, e1001610 (2013).
12. R. R. Brutkiewicz, CD1d Ligands: The Good, the Bad, and the Ugly. J. Immunol. 177, 769-775 (2006).
13. S. Joyce, E. Girardi, D. M. Zajonc, NKT cell ligand recognition logic: molecular basis for a synaptic duet and transmission of inflammatory effectors. J. Immunol. 187, 1081-9 (2011).
14. T. Olszak et al., Microbial Exposure During Early Life Has Persistent Effects on Natural Killer T Cell Function. Science (80-.). 336, 489-493 (2012).
15. I. Brondz, I. Olsen, Multivariate analyses of cellular fatty acids in Bacteroides, Prevotella, Porphyromonas, Wolinella, and Campylobacter spp. J. Clin. Microbiol. 29, 183-9 (1991).
16. E. Miyagawa, R. Azuma, T. Suto, I. Yano, Occurrence of free ceramides in Bacteroides fragilis NCTC 9343. J. Biochem. 86, 311-20 (1979).
17. R. F. Leo, P. L. Parker, Branched-Chain Fatty Acids in Sediments. Science (80-.). 152, 649-650 (1966).
18. D. N. Naik, T. Kaneda, Biosynthesis of branched long-chain fatty acids by species of Bacillus: relative activity of three alpha-keto acid substrates and factors affecting chain length. Can. J. Microbiol. 20, 1701-8 (1974).
19. H. C. Beck, Branched-chain fatty acid biosynthesis in a branched-chain amino acid aminotransferase mutant of Staphylococcus carnosus. FEMS Microbiol. Lett. 243, 37-44 (2005).
20. V. H. Varel, M. P. Bryant, Nutritional features of Bacteroides fragilis subsp. fragilis. Appl. Microbiol. 28, 251-7 (1974).
21. X. Laurent et al., Switching Invariant Natural Killer T (iNKT) Cell Response from Anticancerous to Anti-Inflammatory Effect: Molecular Bases. J. Med. Chem. 57, 5489-5508 (2014).
22. E. Kobayashi, K. Motoki, T. Uchida, H. Fukushima, Y. Koezuka, KRN7000, a novel immunomodulator, and its antitumor activities. Oncol. Res. 7, 529-34 (1995).
23. X. Li et al., Design of a potent CD1d-binding NKT cell ligand as a vaccine adjuvant. Proc. Natl. Acad. Sci. 107, 13010-13015 (2010).
24. D. Sag, P. Krause, C. C. Hedrick, M. Kronenberg, G. Wingender, IL-10-producing NKT10 cells are a distinct regulatory invariant NKT cell subset. J. Clin. Invest. 124, 3725-3740 (2014).
25. T. Olszak et al., Protective mucosal immunity mediated by epithelial CD1d and IL-10. Nature. 509, 497-502 (2014).
26. S. S. Iyer et al., Dietary and Microbial Oxazoles Induce Intestinal Inflammation by Modulating Aryl Hydrocarbon Receptor Responses. Cell. 173, 1123-1134.e11 (2018).
27. L. J. Saubermann et al., Activation of natural killer T cells by alpha-galactosylceramide in the presence of CD1d provides protection against colitis in mice. Gastroenterology. 119, 119-28 (2000).
28. Y. Numata et al., Therapeutic Effect of Repeated Natural Killer T Cell Stimulation in Mouse Cholangitis Complicated by Colitis. Dig. Dis. Sci. 50, 1844-1851 (2005).
29. E. Zigmond et al., Beta-glucosylceramide: a novel method for enhancement of natural killer T lymphoycte plasticity in murine models of immune-mediated disorders. Gut. 56, 82-9 (2007).
30. V. Matyash, G. Liebisch, T. V. Kurzchalia, A. Shevchenko, D. Schwudke, Lipid extraction by methyl-tert-butyl ether for high-throughput lipidomics. J. Lipid Res. 49, 1137-1146 (2008).

### Other Embodiments

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. An isolated compound having the structure of formula (I): 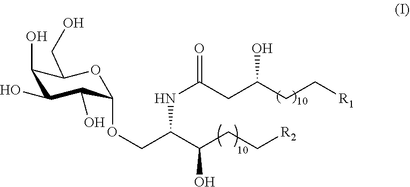
or an enantiomer or diastereomer thereof, wherein:
(b) R₁ and R₂ independently are methyl, ethyl, or branched or unbranched C₃-C₆ alkyl, or
(b) R₁ is methyl, ethyl, or branched or unbranched C₃-C₆ alkyl and R₂ is methyl or branched C₃-C₆ alkyl, or
(c) R₁ is methyl, ethyl, or branched or unbranched C₁-C₅ alkyl and R₂ is methyl, ethyl, or branched or unbranched C₂-C₅ alkyl, or
(d) R₁ is methyl, ethyl, or branched or unbranched C₁-C₅ alkyl and R₂ is methyl or branched C₂-C₅ alkyl.

2. The compound of claim 1, wherein R₂ is a branched alkyl.

3. The compound of claim 1, wherein R₁ is selected from the group consisting of methyl, ethyl, n-propyl, isopropyl, n-butyl, sec-butyl, and n-pentyl.

4. The compound of claim 1 or 3, wherein R₂ is independently selected from the group consisting of methyl, n-propyl, isopropyl, n-butyl, sec-butyl, and n-pentyl.

5. The compound of claim 1, wherein:
R₁ is selected from methyl, ethyl, isopropyl, and sec-butyl; and
R₂ is selected from isopropyl, n-butyl, sec-butyl, and n-pentyl.

6. The compound of claim 1, wherein both of R₁ and R₂ are branched, or R₁ is unbranched and R₂ is branched.

7. The compound of claim 1, wherein the compound is substantially free of homologues, positional isomers, and stereoisomers.

8. The compound of claim 1, wherein the compound is at least 95% pure.

9. The compound of claim 1, wherein R₂ is methyl or branched C₃-C₆ alkyl.

10. The compound of claim 1, wherein R₂ is methyl or branched C₂-C₅ alkyl.

11. A compound having a structure of

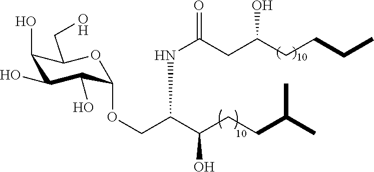

12. A compound having a structure of

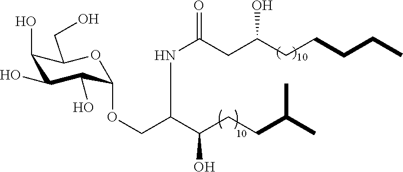

13. A compound having a structure of

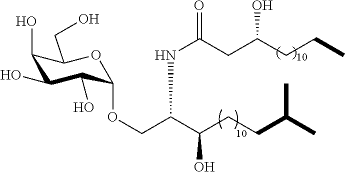

14. A compound having a structure of

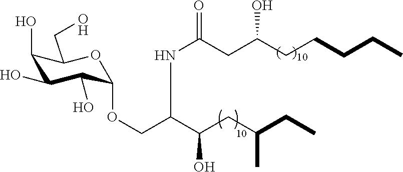

15. A compound having a structure of

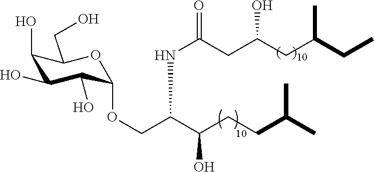

16. A compound having a structure of

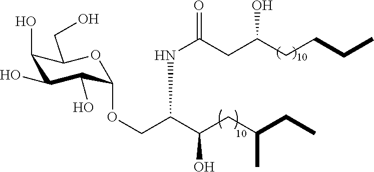

17. A compound having a structure of

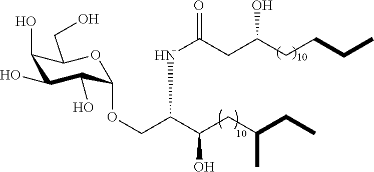

18. A compound having a structure of

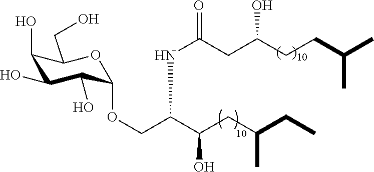

19. A compound having a structure of

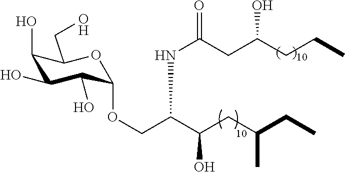

20. A compound having a structure of

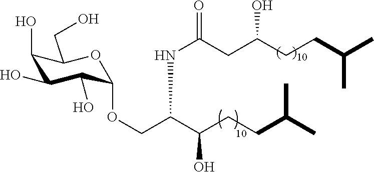

21. The compound of any one of claims 1-20, wherein the fatty acid C3′ position has R-chirality.

22. The compound of any one of claims 1-20, wherein the fatty acid C3′ position has S-chirality.

23. The compound of any one of claims 1-22, wherein the sphinganine C3 position has R-chirality.

24. The compound of any one of claims 1-22, wherein the sphinganine C3 position has S-chirality.

25. The compound of any one of claims 1-24, wherein the compound is at least 80% pure (w/w), or at least 85% pure (w/w), or at least 90% pure (w/w), or at least 95% pure (w/w), or at least 99% pure (w/w), or 100% pure.

26. The compound of any one of claims 1-25, wherein the compound is complexed to an isolated CD1d protein.

27. A composition comprising the compound of any one of claims 1-26.

28. The composition of claim 27, wherein the composition is sterile.

29. A pharmaceutical composition comprising the compound of any one of claims 1-26.

30. The pharmaceutical composition of claim 29, formulated for delivery to gut.

31. The pharmaceutical composition of claim 29, formulated for delivery to lungs.

32. The pharmaceutical composition of any one of claims 29-31, further comprising an additional immunosuppressant.

33. The pharmaceutical composition of claim 29, wherein the compound is formulated as a suppository or an enema.

34. The pharmaceutical composition of claim 29, wherein the compound is formulated as an oral solution or suspension.

35. The pharmaceutical composition of claim 29, wherein the compound is formulated as a capsule, a pill, a lozenge, a tablet,

36. The pharmaceutical composition of claim 35, wherein the compound is formulated as a delayed release capsule, a pill, a lozenge, or a tablet.

37. The pharmaceutical composition of claim 35, wherein the compound is formulated as an extended release capsule, a pill, a lozenge, or a tablet.

38. The pharmaceutical composition of claim 36, wherein the compound is formulated as a capsule, a pill, a lozenge, or a tablet, having an enteric coating.

39. The pharmaceutical composition of claim 29, wherein the compound is formulated in a pH-dependent controlled release capsule, a pill, a lozenge, or a tablet.

40. A method comprising
administering to a subject having or at risk of developing a condition characterized by increased NKT cell numbers or activity a compound of any one of claims 1-10 or A1-A15 in an effective amount to decrease NKT cell numbers or activity in the subject.

41. The method of claim 40, wherein the condition is an autoimmune disease.

42. The method of claim 40, wherein the condition is inflammatory bowel disease.

43. The method of claim 40, wherein the condition is colitis.

44. The method of claim 40, wherein the condition is asthma.

45. The method of any one of claims 40-43, wherein the compound is administered to gut of the subject.

46. The method of any one of claims 40-43, wherein the compound is administered to lungs of the subject.

47. The method of claim 40, wherein the condition is lupus.

48. The method of claim 40, wherein the condition is multiple sclerosis.

49. The method of claim 40, wherein the condition is arthritis.

50. The method of claim 40, wherein the condition is inflammatory dermatitis.

51. The method of any one of claims 40-50, wherein the compound is administered locally.

52. The method of any one of claims 40-51, wherein the subject is human.

53. The method of any one of claims 40-52, wherein the subject is administered another immunosuppressant.

54. The method of any one of claims 40-53, further comprising identifying the subject as having or at risk of developing the condition.

55. The method of any one of claims 40-54, wherein the subject is less than 5 years of age, or less than 1 year of age, or less than 6 months of age.

56. The method of any one of claims 40-54, wherein the subject is pregnant or is a female of child-bearing age.

57. The method of any one of claims 40-56, wherein the compound is administered with an isolated CD1d protein.

58. The method of any one of claims 40-57, wherein the compound is administered in a complex with CD1d protein.

59. A method comprising
contacting antigen presenting cells with the compound of any one of claims 1-26 and
contacting the antigen presenting cells with activated NKT cells.

60. The method of claim 59, wherein the antigen presenting cells are dendritic cells.

61. The method of claim 59 or 60, wherein the antigen presenting cells are contacted with the compound in vitro.

62. The method of claim 59, 60 or 61, wherein the antigen presenting cells, loaded with the compound, are contacted with the activated NKT cells in vivo.

63. A method comprising
contacting the compound of any one of claims 1-25 with CD1d protein to form an alpha-GC-CD1d complex, and
contacting the alpha-GC-CD1d complex with activated NKT cells.

64. The method of claim 63, wherein the contacting occurs in vitro.

65. The method of claim 63, wherein the contacting occurs in vivo.

66. The method of claim 63, 64 or 65, wherein the CD1d protein is a CD1d tetramer.

67. The method of claim 63, 64, 65 or 66, further comprising administering the activated NKT cells, after contact with the alpha-GC-CD1d complex, to a subject having or at risk of developing a condition characterized by increased NKT cell numbers and/or activity.

68. A method of making a compound of formula (I) according to any one of claims 1-5, comprising:
(1) condensing a compound of formula (II): 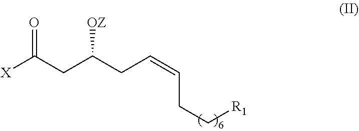
wherein:
X is —OH or a leaving group; and
Z is an oxygen protecting group;
with a compound of formula (III): 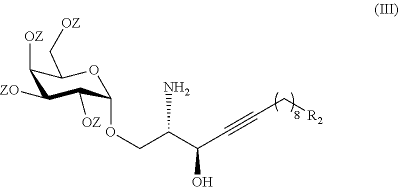
wherein:
each Z independently is an oxygen protecting group;
to afford a compound of formula (IV): 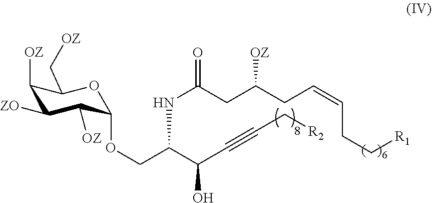
and;
(2) reducing the unsaturated carbon-carbon bonds and deprotecting the protected hydroxyl groups to afford the compound of formula (I).

69. The method of claim 68, wherein X is —OH.

70. The method of claim 68, wherein each Z is Bn.

71. The method of claim 68, wherein step (1) comprises contacting the compound of formula (II) with the compound of formula (III) in the presence of a dehydration agent.

72. The method of claim 71, wherein the dehydration agent is a carbodiimide.

73. The method of claim 68, wherein step (2) comprises contacting the compound of formula (IV) with H₂ in the presence of a catalyst.

74. The method of claim 73, wherein the catalyst comprises palladium.

75. The method of claim 68, wherein the method makes a plurality of compounds of formula (I), and comprises:
(1) condensing a plurality of compounds of formula (II) with a plurality of compounds of formula (III), to afford a plurality of compounds of formula (IV); and
(2) reducing the unsaturated carbon-carbon bonds and deprotecting the protected hydroxyl groups of the plurality of compounds of formula (IV) to afford the plurality of compounds of formula (I).
